(19) United States
(12) Patent Application Publication
(10) Pub. No.: US 2016/0251410 A1
(43) Pub. Date: Sep. 1, 2016

(54) COMPOSITIONS AND METHODS FOR EXPRESSING RECOMBINANT POLYPEPTIDES

(71) Applicants: YISSUM RESEARCH DEVELOPMENT COMPANY OF THE HEBREW UNIVERSITY OF JERUSALEM LTD., Jerusalem (IL); TEL HASHOMER MEDICAL RESEARCH INFRASTRUCTURE AND SERVICE LTD., Ramat-Gan (IL)

(72) Inventors: Boaz TIROSH, Mevasseret Zion (IL); Maya DADIANI, Rehovot (IL)

(73) Assignees: Yissum Research Development Company of the Hebrew University of Jerusalem Ltd., Jerusalem (IL); Tel Hashomer Medical Research Infrastructure and Services Ltd., Rama-Gan (IL)

(21) Appl. No.: 14/914,003

(22) PCT Filed: Sep. 3, 2014
(86) PCT No.: PCT/IL2014/050793

§ 371 Date: Feb. 24, 2016

Related U.S. Application Data
(60) Provisional application No. 61/873,202, filed on Sep. 3, 2013.

(51) Int. Cl. C07K 16/00

(52) U.S. Cl. CPC C07K 16/00; C07K 2317/14

(57) ABSTRACT

Methods of expressing a recombinant polypeptide of interest are provided. Accordingly there is provided a method comprising providing a cell having been contacted with an agent which downregulates an expression of a tuberous sclerosis (TSC) protein or directly inhibits an activity of same; and contacting the cell with a polynucleotide encoding the recombinant polypeptide of interest. Also provided is a method comprising contacting a cell with an agent which downregulates expression of a tuberous sclerosis (TSC) protein or directly inhibits an activity of same; and a polynucleotide encoding the recombinant polypeptide of interest. Also provided are isolated cells, cell cultures and articles of manufacture for recombinant expression of a recombinant polypeptide of interest.

### FIELD AND BACKGROUND OF THE INVENTION

The present invention, in some embodiments thereof, relates to compositions and methods for expressing recombinant polypeptides.

Recombinant therapeutic proteins and antibodies play an important role in treatment of a large variety of diseases. Monoclonal antibodies, for example, are currently used to treat millions of patients suffering from various autoimmune and inflammatory diseases worldwide and have become the blockbuster products of today's biopharmaceutical manufacturing industry. It is estimated that about 30% of the new coming drugs are likely to be based on antibodies in the next decade. Thirty recombinant antibodies and Fc fusion polypeptides were approved for marketing with sales in 2008 that reached 35 billion dollars. Mammalian cell expression systems are the dominant tool today for producing complex biotherapeutic proteins. A restricted list of cell lines is approved by the FDA for use in recombinant protein production, amongst which the Chinese hamster ovary (CHO) cells are the most commonly used.

In order to meet market demand and improve production capacity, a vast research has been performed to optimize the production process, such as the development of fed-batch cultures, and to increase the level of expression and secretion of the recombinant protein. Research has been focused on development of gene expression technology to increase recombinant gene copy number or transcriptional activity, and on genetic engineering of the cells to increase viability, cell growth and production including over expression of proteins involved in modulating signaling such as BLIMP-1, initiation of ER expansion such as XBP-1, ATF6 and inhibition of apoptotic cell death such as the survival proteins Bcl-2 or Bcl-xL.

Mammalian target of Rapamycin (mTOR) is a key metabolic serine/threonine kinase known to modulate many cellular activities including translation control, ribosome biogenesis, apoptosis modulation, cell cycle regulation, metabolic modulation, neuronal function and autophagy. Basically, the mTOR funnels multiple growth-permitting inputs and growth-promoting outputs on both cellular and organism levels, thereby maintaining homeostasis and properly coordinating growth with nutrient conditions [Arsham and Neufeld, Current Opinion in Cell Biology (2006) 18:589-597].

Briefly, the mTOR pathway is divided into upstream and downstream phosphorylation cascades, revolving around the mTOR protein which is found in at least two different complexes, referred to as mTOR complex 1 (mTORC1) and mTOR complex 2 (mTORC2). mTORC1 is inhibited by Rapamycin and directly controls translational activity, while mTORC2 is less sensitive to Rapamycin and presumably has no direct implication on translation, although it was recently proposed to affect mTORC1 activity through the Akt pathway [Foster and Fingar, J Biol Chem (2010) 285:14071-77]. When activated, mTORC1 promotes anabolic processes and enhances protein synthesis and cell growth (8). On the contrary, when mTORC1 is inhibited translation is reduced, growth is arrested and apoptosis and macroautophagy are induced [(9) and Ozcan et al. Mol Cell (2008) 29(5):541-551]. mTOR, primarily in the form of mTORC1, plays major roles in cancer and immune functions (10, 11). Much of the knowledge on the role of mTOR in immune regulation has been obtained from loss of function experiments using Rapamycin or analogs thereof; however, the effect that mTOR activation has on the immune system remains unclear.

Among many other functions, the mTOR pathway adjusts protein synthesis to the well being of the cell, for example mTOR is activated when the ATP:AMP ratio or the intracellular pool of amino acids are high. The control of protein synthesis is regulated by mTOR-specific phosphorylation of 4E binding protein (4E-BP) and ribosomal protein S6 kinase 1 (S6), which, when phosphorylated, mediate accelerated protein synthesis and cell growth (12-14). Hence, inhibition of the mTOR globally reduces protein synthesis and cell size.

A wide array of mTOR inputs from multiple intracellular and environmental stimuli have been identified, including amino acids, oxygen, AMP/ATP ratio and growth factors, as well as the regulatory proteins that facilitate their effects on mTOR. Such proteins include AMPK, Rheb and the tumor suppressors LKB1, p53, and tuberous sclerosis complex (TSC) 1/2. [Arsham and Neufeld, Current Opinion in Cell Biology (2006) 18:589-597].

The tuberous sclerosis complex (TSC), a complex that contains TSC1 and TSC2 tumor suppressor genes, acts as a negative regulator of mTOR. TSC is one of the most well established upstream regulators of mTORC1, acting as a molecular switchboard that integrates several incoming environmental signals. Loss of TSC function either by TSC1 or TSC2 deficiency leads to constitutive activation of mTOR resulting in the development of tumors and neurological disorders. At the cellular level it has been shown that deletion of TSC1 or TSC2 leads to uncontrolled protein synthesis, development of endoplasmic reticulum (ER) stress, activation of the unfolded protein response (UPR), severe insulin/IGF-1 resistance and apoptosis [e.g. Ozcan et al. Mol Cell (2008) 29(5):541-551].

At the mature state of B cell development, mTOR is activated in response to toll-like receptor and B cell receptor (BCR) ligation down-stream to the PI3K/Akt signaling pathway. Akt activates mTORC1 indirectly by reversing the TSC inhibition of mTOR. It was previously reported that mTOR is the predominant mechanism that controls protein synthesis in the late phase of LPS-activated B cells, in a manner rigorously controlled by ER stress. ER stress is a state of imbalance between the protein-folding capacities and the amount of proteins in the ER. A network of signaling pathways termed the Unfolded Protein Response (UPR) restores the disrupted balance in the ER. In mammalian cells the UPR operates in three parallel pathways, named after the sensors of ER stress: IRE1, PERK and ATF6. The sensors activate downstream signals that regulate gene transcription and protein synthesis (1). Following a signal to differentiate into plasma cells (PC), the ER of a B cell expands and facilitates synthesis, proper folding, assembly and secretion of copious amounts of antibodies. The remodeling of the ER in the course of PC differentiation is controlled solely by the IRE1/XBP-1 pathway of the UPR (2, 3). In the absence of XBP-1 or IRE1, B cells develop normally to the mature state, but yield long-lived PCs that secrete small amounts of immunoglobulins (Igs) (4-6).

Deletion of TSC1 in some cell types was shown to induce UPR (13) and thus may contribute indirectly to PC development. It has also been shown that B cells knocked-out for TSC1 exhibit impaired development, enhanced apoptosis of developing PCs, loss of the marginal zone subset and defects in germinal centers; however, serum antibody titers are normal.(15,16)

### ADDITIONAL RELATED ART

Dreesen and Fussenegger;

Edros et al. [BMC Biotechnology (2014) 14:15-24];

Lee and Lee [Biotechnolo. Bioeng. (2012) 109: 3093-3102];

Balcarcel and Stephanopoulos [Biotechnol. Bioeng. (2001) 76(1): 1-10];

Chong et al. [American Institute of Chemical Engineers Biotechnol. Prog. (2009) 25: 866-873]; and

Hara et al. [J Biol Chem. (1998) 273(23): 14484-94].

### SUMMARY OF THE INVENTION

According to an aspect of some embodiments of the present invention there is provided a method of expressing a recombinant polypeptide of interest, the method comprising: providing a cell having been contacted with an agent which downregulates an expression of a tuberous sclerosis (TSC) protein or directly inhibits an activity of same; and contacting the cell with a polynucleotide encoding the recombinant polypeptide of interest.

According to an aspect of some embodiments of the present invention there is provided a method of expressing a recombinant polypeptide of interest, the method comprising contacting a cell with:

(i) an agent which downregulates expression of a tuberous sclerosis (TSC) protein or directly inhibits an activity of same;

(ii) a polynucleotide encoding the recombinant polypeptide of interest.

According to an aspect of some embodiments of the present invention there is provided a method of increasing production of a recombinant polypeptide of interest, the method comprising contacting a cell which comprises a polynucleotide encoding the recombinant polypeptide of interest with an agent which downregulates an expression of a tuberous sclerosis (TSC) protein or directly inhibits an activity of same, thereby increasing production of the recombinant polypeptide of interest.

According to some embodiments of the invention, contacting (i) and (ii) are performed concomitantly.

According to some embodiments of the invention, contacting (i) and (ii) are performed sequentially.

According to some embodiments of the invention, contacting (i) is performed prior to contacting (ii).

According to some embodiments of the invention, contacting (ii) is performed prior to contacting (i).

According to an aspect of some embodiments of the present invention there is provided an isolated cell having been contacted with an agent which downregulates an expression of a tuberous sclerosis (TSC) protein or directly inhibits an activity of same, wherein the cell further comprises a modified carbohydrate synthesis pathway, glutamine synthetase (GS) and/or dihydrofolate reductase (DHFR) as compared to a control cell of the same species.

According to some embodiments of the invention, the method further comprising isolating the recombinant polypeptide.

According to some embodiments of the invention, the method further comprising contacting the cell with an agent which downregulates an activity and/or expression of a pro-apoptotic gene.

According to some embodiments of the invention, the method further comprising contacting the cell with an agent which upregulates an activity and/or expression of an anti-apoptotic gene.

According to an aspect of some embodiments of the present invention there is provided an isolated cell obtainable according to the method as described herein.

According to an aspect of some embodiments of the present invention there is provided an article of manufacture identified for recombinant expression of a recombinant polypeptide of interest comprising a packaging material packaging an agent for down regulating expression of a tuberous sclerosis (TSC) protein or directly inhibiting an activity of same; and a nucleic acid construct for expressing the polypeptide of interest.

According to an aspect of some embodiments of the present invention there is provided an article of manufacture identified for recombinant expression of a recombinant polypeptide of interest comprising a packaging material packaging an isolated cell having been contacted with an agent which downregulates an expression of a tuberous sclerosis (TSC) protein or directly inhibits an activity of same, wherein the cell further comprises a modified carbohydrate synthesis pathway, glutamine synthetase (GS) and/or dihydrofolate reductase (DHFR) as compared to a control cell of the same species.

According to some embodiments of the invention, the article of manufacture of further comprising an agent for down regulating an activity and/or expression of a pro-apoptotic gene.

According to some embodiments of the invention, the article of manufacture further comprising an agent for up regulating an activity and/or expression of an anti-apoptotic gene.

According to an aspect of some embodiments of the present invention there is provided an isolated cell comprising an exogenous agent which downregulates expression of a tuberous sclerosis (TSC) protein or directly inhibits an activity of same and expressing a recombinant polypeptide of interest.

According to some embodiments of the invention, the isolated cell further comprising an exogenous agent which downregulates an activity and/or expression of a pro-apoptotic gene.

According to some embodiments of the invention, the isolated cell further comprising an exogenous agent which upregulates an activity and/or expression of an anti-apoptotic gene.

According to some embodiments of the invention, the pro-apoptotic gene is selected from the group consisting of BAX, BAK and PUMA.

According to some embodiments of the invention, the anti-apoptotic gene is selected from the group consisting of Bcl-2, Bcl-xL, Bcl-w, Mcl-1 and XIAP.

According to some embodiments of the invention, the cell is a mammalian cell.

According to some embodiments of the invention, the mammalian cell is selected from the group consisting of a Chinese Hamster Ovary (CHO), HEK293, PER.C6, HT1080, NS0, Sp2/0, BHK, Namalwa, COS, HeLa and Vero cell.

According to some embodiments of the invention, the mammalian cell comprises a Chinese Hamster Ovary (CHO) and HEK293 cell.

According to some embodiments of the invention, the polypeptide is a secreted polypeptide.

According to some embodiments of the invention, the polypeptide comprises an antibody or an antibody fragment.

According to some embodiments of the invention, the antibody or antibody fragment is a probody.

According to some embodiments of the invention, the polypeptide is selected from the group consisting of CTLA4-Ig, IFNβ, IFNγ, TNFα and IL-6.

According to some embodiments of the invention, the recombinant polypeptide is a human recombinant polypeptide.

According to some embodiments of the invention, the isolated cell being a cell line.

According to some embodiments of the invention, there is provided a cell culture comprising the isolated cell as described herein and a cell culture medium.

According to an aspect of some embodiments of the present invention there is provided a method of selecting an agent which downregulates expression of a tuberous sclerosis (TSC) protein or directly inhibits an activity of same, the method comprising:

(a) contacting a population of cells expressing a reporter polypeptide with an agent putative for down regulating expression of a TSC protein or directly inhibiting an activity of same; and

(b) determining whether expression and/or secretion of the polypeptide increases following the contacting with the agent;

wherein an increase above a predetermined threshold indicates the agent downregulates expression of a TSC protein or directly inhibits an activity of same.

According to some embodiments of the invention, the determining is effected by flow cytometry, western blot and/or ELISA.

According to some embodiments of the invention, the contacting is effected ex-vivo or in-vitro.

According to some embodiments of the invention, the agent is a polynucleotide.

According to some embodiments of the invention, the agent is a RNA silencing agent.

According to some embodiments of the invention, the agent is a site specific recombinase.

According to some embodiments of the invention, the agent is an engineered endonuclease for genome editing.

According to some embodiments of the invention, the polynucleotide is selected from the group consisting of an antisense, siRNA, miRNA, zinc finger nuclease, CRISPR/Cas and TALEN.

According to some embodiments of the invention, the agent comprises a polynucleotide sequence selected from the group consisting of SEQ ID NOs. 20-28.

According to some embodiments of the invention, the agent interferes with the formation of a TSC1/TSC2 complex.

According to some embodiments of the invention, the agent binds to and/or cleaves the TSC.

According to some embodiments of the invention, the agent is selected from the group consisting of an aptamer, a small molecule, an inhibitory peptide, antibody and antibody fragment.

According to some embodiments of the invention, the agent increases phosphorylation of S6.

According to some embodiments of the invention, the tuberous sclerosis is TSC1.

According to some embodiments of the invention, the tuberous sclerosis is TSC2.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph representing antibodies titers in the sera of wild-type (wt), CD19-Cre/XBP1^{f/f} (XBP-1 KO), CD19-Cre/TSC1^{f/f} (TSC1 KO) and CD19-Cre/XBP1^{f/f}/TSC1^{f/f} (DKO) mice as analyzed by ELISA. Error bars represent SE, (n=10).

FIGS. 2A-B show YFP reporter expression in the deletion of TSC1 and XBP-1 in CD19-Cre/ROSA26-floxed stop-lacZ YFP (DKO/YFP) mice. APRIL-stimulated mesenteric lymph node (MLN) cells isolated from DKO/YFP mice were stained and sorted for YFP⁺B220⁺ and YFP⁻B220⁺ cells. FIG. 2A is a representative western blot photograph demonstrating reduced expression of TSC1 protein in YFP⁺B220⁺ cells as compared to YFP⁻B220⁺ cells. FIG. 2B is a representative PCR photograph demonstrating recombination of the floxed XBP-1 allele in YFP⁺B220⁺ cells. (n=2 independent experiments).

FIGS. 3A-C are graphs demonstrating the effect of TCS1 on B cell maturation and differentiation in wt/YFP, CD19-Cre/XBP1^{f/f}/ROSA26-floxed stop-lacZ YFP (XBP-1 KO/YFP), CD19-Cre/TSC1^{f/f}/ROSA26-floxed stop-lacZ YFP (TSC-1 KO/YFP) and CD19-Cre/XBP1^{f/f}/TSC1^{f/f}/ROSA26-floxed stop-lacZ YFP (DKO/YFP) mice. FIG. 3A shows representative flow cytometry dot plots of B220 vs. YFP expression in cells isolated from spleens, peripheral lymph nodes (pLN) and bone marrow (BM) of the various mouse strains. FIG. 3B shows representative flow cytometry dot plots of B220 vs. CD138 expression in cells isolated from pLN (upper panel) and BM (lower panel) of the various mouse strains. FIG. 3C shows bar graphs representing the percentages of YFP⁺CD138⁺ cells relative to the total YFP⁺ cells from pLN (upper panel) and BM (lower panel) of the various mouse strains, as evaluated by flow cytometry.

FIG. 4 is a transmission electron microscopy photograph (Magnification ×9700) of YFP⁺CD138⁺ cells sorted from BM of wt/YFP, XBP-1 KO/YFP, TSC-1 KO/YFP and DKO/YFP mice demonstrating ER morphology in plasma cells (PCs) of the different strains.

FIG. 5 shows representative flow cytometry dot plots of CD138 vs. YFP expression in APRIL-stimulated MLN cells isolated from wt/YFP, XBP-1 KO/YFP, and DKO/YFP mice demonstrating significant reduction in the proportion of YFP population compared to the YFP⁻ population, however an increased percentage of YFP⁺CD138⁺ cells relative to the total YFP⁺ population in DKO/YFP mice.

FIG. 6 shows representative flow cytometry dot plots of APRIL-stimulated MLN cells isolated from wt/YFP, XBP-1 KO/YFP, and DKO/YFP mice Demonstrating that most of the YFP⁻ cells express CD5. In the upper panel are dot plots of CD5 vs. YFP expression and in the lower panel are dot plots of intracellular kappa light chain content vs. YFP expression in B220⁺ gated cells.

FIG. 7 is a bar graph representing immunoglobulin (Ig) levels in the culture supernatants of APRIL-stimulated MLN cells isolated from wild-type (wt), CD19-Cre/XBP1^{f/f} (XBP-1 KO), CD19-Cre/XBP1^{f/f}/TSC1^{f/f} (DKO) mice, as evaluated by ELISA (n=6). Error bars indicate SE.

FIG. 8 shows the number of antibody forming cells in YFP⁺ cells sorted following APRIL stimulation of MLN isolated from wt/YFP, XBP-1 KO/YFP, TSC-1 KO/YFP and DKO/YFP mice, as evaluated by IgA ELISPOT (n=3).

FIG. 9 shows pulse chase analysis performed on equal number of YFP⁺ cells sorted following APRIL stimulation of MLN isolated from wt/YFP, XBP-1 KO/YFP and DKO/YFP mice. In the upper panel are photographs of anti-Ig immunoprecipitation. In the lower panel a bar graph showing quantification of synthesized Ig at the end of the pulse and secreted Ig following 4 hours (n=3) demonstrating increased synthesis and secretion of Ig in cells obtained from DKO/YFP as compared to XBP-1 KO/YFP mice. Error bars indicate SE.

FIG. 10 is a bar graph demonstrating the effect of Rapamycin on Ig secretion to the culture supernatant by APRIL-stimulated DKO/YFP MLN cells, as analyzed by ELISA. Error bars indicate SE, (n=4).

FIG. 11 shows representative flow cytometry histograms demonstrating that deletion of TSC1 does not affect B cell proliferation. Splenic B cells were isolated from RERT/wild-type (wt), RERT/XBP-1^{f/f} (XBP-1 KO), RERT/TSC1^{f/f} (TSC1 KO) and RERT/XBP-1^{f/f}/TSC1^{f/f} (DKO) mice following tamoxifen administration, labeled with CFSE and either analyzed by flow cytometry (day 0) or stimulated with LPS before flow cytometry analysis (day 3).

FIG. 12 shows representative flow cytometry dot plots of propidium iodide (PI) vs. CD138 expression (lower panel) in LPS-stimulated splenic B cells isolated from RERT/wild-type (wt), RERT/XBP-1^{f/f} (XBP-1 KO) and RERT/XBP-1^{f/f}/TSC1^{f/f} (DKO) mice following tamoxifen administration, demonstrating that deletion of TSC1 induces apoptosis following stimulation. Cells were gated according to side scatter (SSC) vs. forward scatter (FSC) dot plots as shown in the upper panel.

FIGS. 13A-B demonstrate that TSC1 deletion promotes Ig secretion. FIG. 13A is a bar graph of Ig levels in the supernatants of LPS-stimulated splenic B cells isolated from RERT/wild-type (wt), RERT/XBP-1^{f/f} (XBP-1 KO) and RERT/XBP-1^{f/f}/TSC1^{f/f} (DKO) mice following tamoxifen administration (n=4), as evaluated by ELISA. FIG. 13B demonstrates IgM synthesis and secretion by LPS-stimulated splenic B cells isolated from RERT/wild-type (wt), RERT/XBP-1^{f/f} (XBP-1 KO) and RERT/XBP-1^{f/f}/TSC1^{f/f} (DKO) mice following tamoxifen administration, as evaluated by pulse chase analysis. Error bars indicate SE.

FIGS. 14A-B demonstrate the expression of Ly6C in APRIL-stimulated MLN cells isolated from wt/YFP, XBP-1 KO/YFP, TSC-1 KO/YFP and DKO/YFP mice. FIG. 14A is a bar graph representing LyC6 mRNA levels in YFP⁺ cells sorted 6 days following stimulation. FIG. 14B shows flow cytometry dot plots of LyC6 vs. CD138 expression on YFP+ gated cells on days 0 and 6 following stimulation.

FIG. 15 is a bar graph of IgA levels in the supernatants of MLN cells of XBP-1 KO/YFP, and DKO/YFP 6 days following stimulation with APRIL in the presence or absence of a LyC6 blocking antibody, as evaluated by ELISA. Error bars indicate SE (n=3)

FIGS. 16A-B demonstrate that blockade of Ly6C1 does not affect viability and differentiation into PCs following APRIL stimulation. FIG. 16A shows representative flow cytometry dot plots of SSC vs. FSC in MLN cells of XBP-1 KO/YFP, and DKO/YFP 6 days following stimulation with APRIL in the presence or absence of a LyC6 blocking antibody. The gate represents live cells. FIG. 16B shows flow cytometry dot plots of FSC vs. CD138 expression in the gated live cells presented in FIG. 16A.

FIGS. 17A-B shows flow cytometry dot plots of GFP expression vs. FSC demonstrating the percentages of CHO (FIG. 17A) or HEK293 (FIG. 17B) cells stably expressing the recombinant GFP-Fc following transfection and sorting.

FIGS. 18A-B demonstrate high level of expression and secretion of the recombinant GFP-Fc by transfected HEK293 cells. FIG. 18A is western blot photograph demonstrating specific expression of GFP-Fc using anti-GFP antibody. FIG. 18B demonstrates pulse chase analysis performed on HEK293 transfected cells. In the upper panel are photographs of intracellular GFP and in the lower panel are photographs of secreted GFP.

FIG. 19 shows the analysis for CRISPR vector clones for down regulating TSC2 expression. Shown is a poly acryl amide (PAGE) gel photograph of CRISPR vector (pX330) following ligation of the gRNA sequences and digestion with NdeI. Arrows indicate two positive insertion clones.

FIGS. 20A-B demonstrate the effect of down-regulating TSC2 expression using the CRISPR system in HEK293T GFP-Fc cells. FIG. 20A shows flow cytometry histograms demonstrating no significant change in the cells forward scatter (FSC) and an increase in the levels of expression of recombinant GFP-Fc as well as the cells side scatter (SSC) in the transfected cells. FIG. 20B shows western blot photographs demonstrating reduced TSC2 protein levels in the transfected cells.

FIG. 21 shows GFP-Fc secretion by HEK293 GFP-Fc cells transfected with TSC2 CRISPR vector, as evaluated by fluorescent scanner.

FIG. 22 demonstrates the correlation between deletion of TSC2 and increased GFP-Fc synthesis in HEK293 cells. Shown are histograms and bar graphs of GFP expression as evaluated by flow cytometry and western blot photograph demonstrating specific expression of GFP-Fc using anti-GFP antibody in HEK293 GFP-Fc clones 2E, 3F, 4D and 4E transfected with TSC2 CRISPR vector.

FIG. 23 shows western blot photographs demonstrating reduced TSC2 protein levels and increased phosphorylated S6 levels by CHO GFP-Fc cells transfected with TSC2 CRISPR vector.

FIG. 24 shows flow cytometry dot plots of SSC vs. PI in CHO-GFP-Fc and their TSC2 KO derivatives at the indicated time intervals demonstrating that TSC2 KO does not affect cell viability.

### DESCRIPTION OF SPECIFIC EMBODIMENTS OF THE INVENTION

The present invention, in some embodiments thereof, relates to compositions and methods for expressing recombinant polypeptides.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Recombinant therapeutic proteins and antibodies in particular play an important role in treatment of a large variety of diseases. Mammalian cell expression systems are the dominant tool today for producing complex biotherapeutic proteins.

The mTOR pathway is complicated, funneling a number of upstream and downstream signaling pathways. While ectopic overexpression of human mTOR was shown to increase proliferation, viability and secretion of recombinant antibody and glycoprotein in CHO cells [Dreesen and Fussenegger; Biotechnol. Bioeng. (2011) 108: 853-866], other studies have found that treatment with Rapamycin, a mTOR inhibitor, delayed apoptosis and enhanced secretion of recombinant antibody in CHO and mouse hybridma cells [Lee and Lee, Biotechnolo. Bioeng. (2012) 109: 3093-3102; Balcarcel and Stephanopoulos, Biotechnol. Bioeng. (2001) 76(1): 1-10, respectively]. Another study by Chong et al. [American Institute of Chemical Engineers Biotechnol. Prog. (2009) 25: 866-873] has shown that adenosine treatment of CHO cells expressing human IFNg causes growth arrest, activates AMPK and on the other hand increases ATP levels leading to increased production of recombinant IFNg by an overall increase in mTOR activity. Hence present studies of the mTOR pathway are divided with respect to its role in recombinant protein production.

The role of TSC in cells growth and productivity has never been disclosed. On the contrary, previous work by Edros et al. [BMC Biotechnology (2014) 14:15-24] indicated no significant expression of mTOR mRNA in antibody producing CHO cells and no significant difference in TSC1 nor TSC2 mRNA expression in high antibody producing CHO cells compared to low antibody producing CHO cells.

Whilst reducing the present invention to practice, the present inventor has now uncovered that deletion of TSC results in increased production and secretion of antibodies by B cells and increase production and secretion of a recombinant protein by CHO and HEK293 cells and suggests that downregulating expression and/or activity of TSC may serve as a general strategy to enhance protein production by a cell.

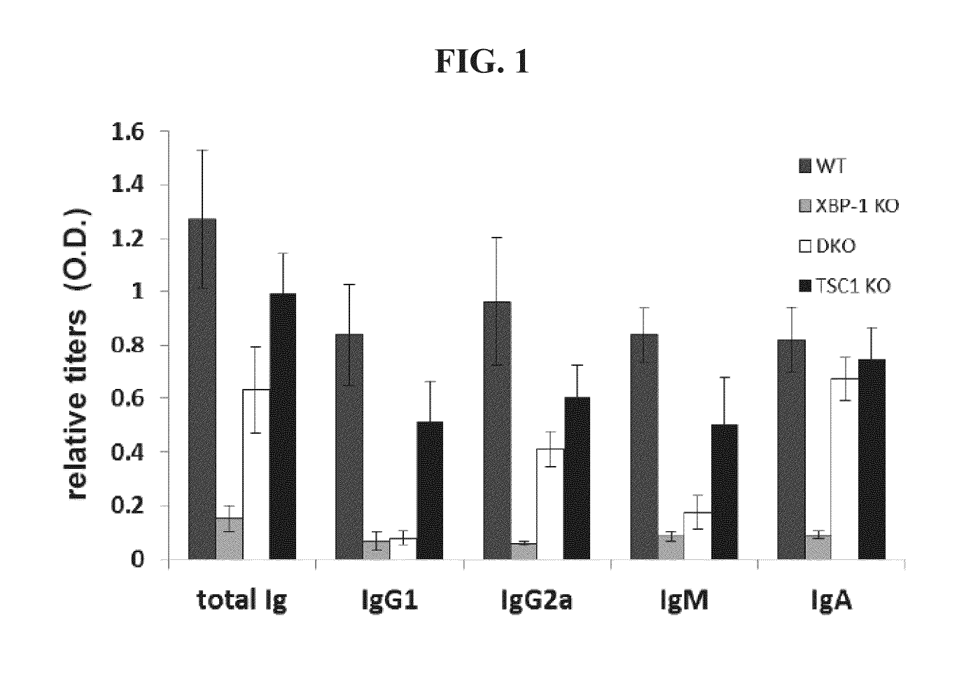

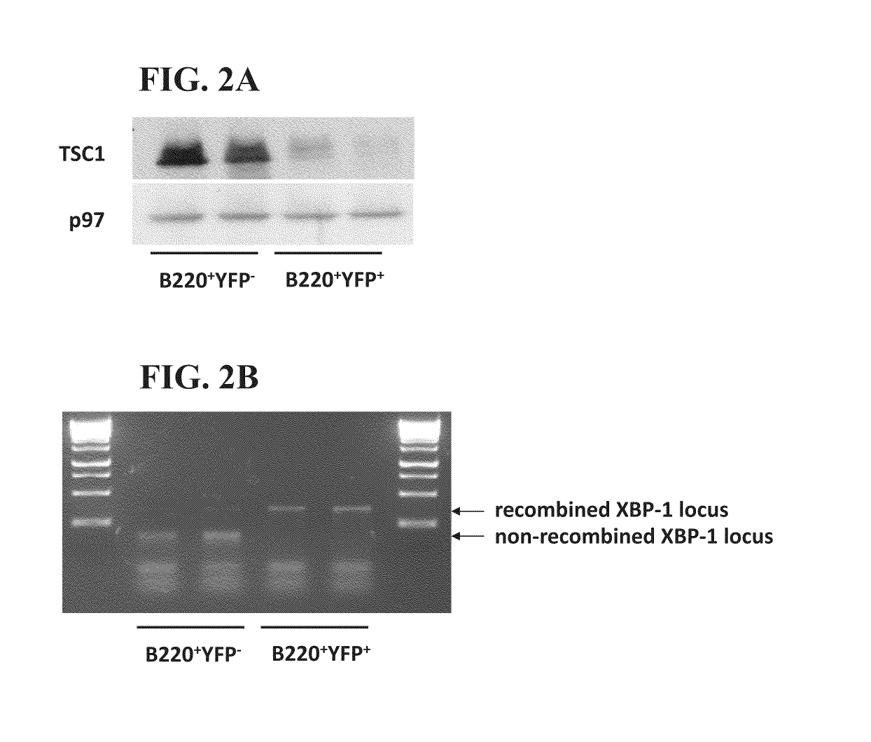

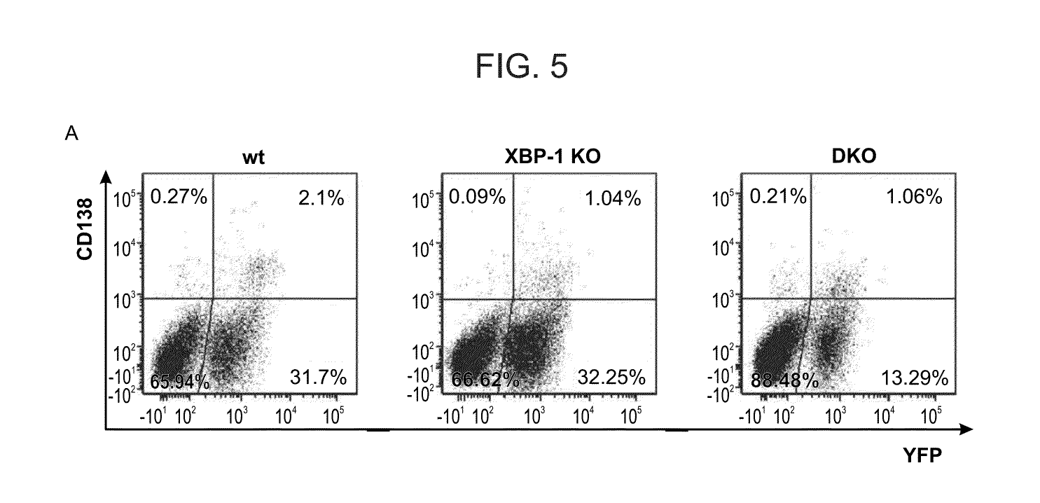

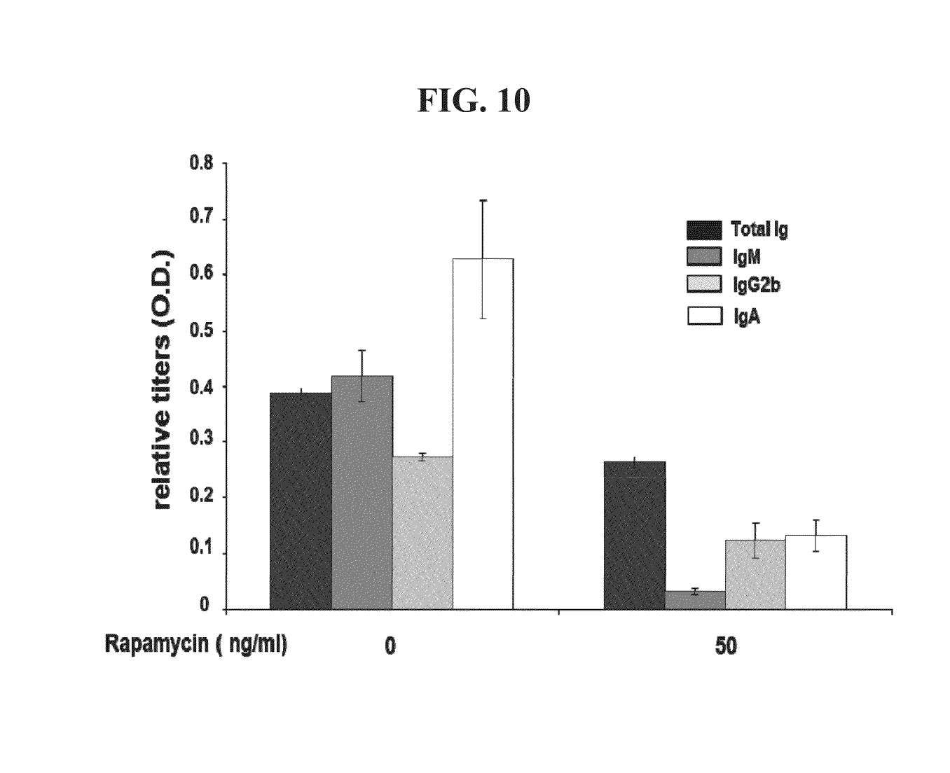

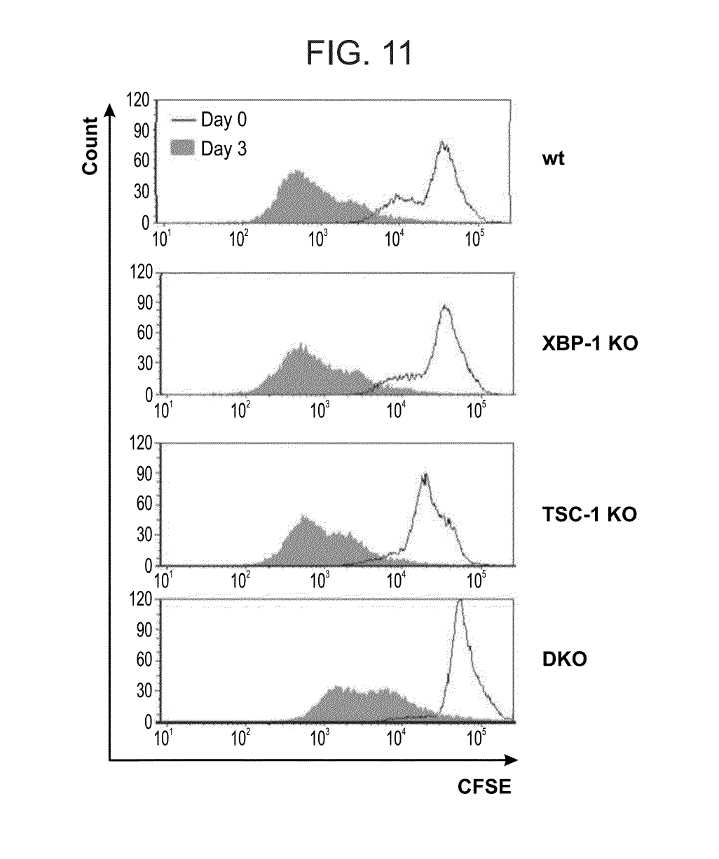

As is illustrated hereinunder and in the examples section, which follows, the present inventor have shown that TSC deletion promotes differentiation of B cells into plasma cells (PCs, Example 1, FIGS. 1, 2A-B, 3A-C and 4). Following stimulation with either APRIL (a TNF family member cytokine) or LPS, TSC deletion results in reduced viability of B cells however the surviving cells are enriched in PCs which possess a better secretory capacity due to increased immunoglobulin synthesis and expression of specific elements in the PC program, such as Ly6C. Strikingly, these activities do not require the IRE1/XBP-1 arm of the UPR (Example 1, FIGS. 5-9, 12, 13A-B, 14A-B, 15 and 16A-B). Even more, the observed increase in immunoglobulin secretion may be attributed to mTOR activation as addition of Rapamycin reduced the levels of secretion (Example 1, FIG. 10). In addition, it seems that deletion of TSC1 has no effect on proliferation of B cells in response to stimulation (Example 1, FIG. 11).

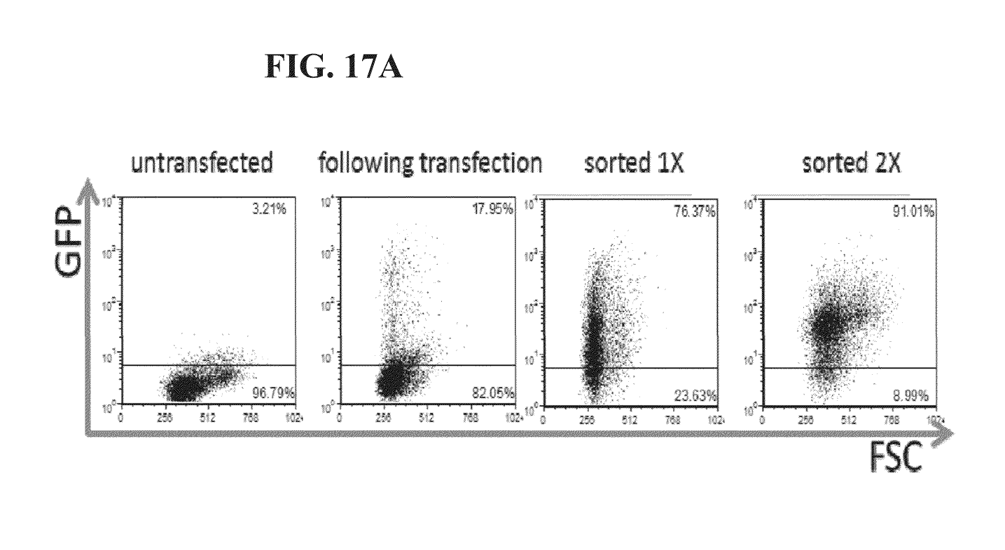

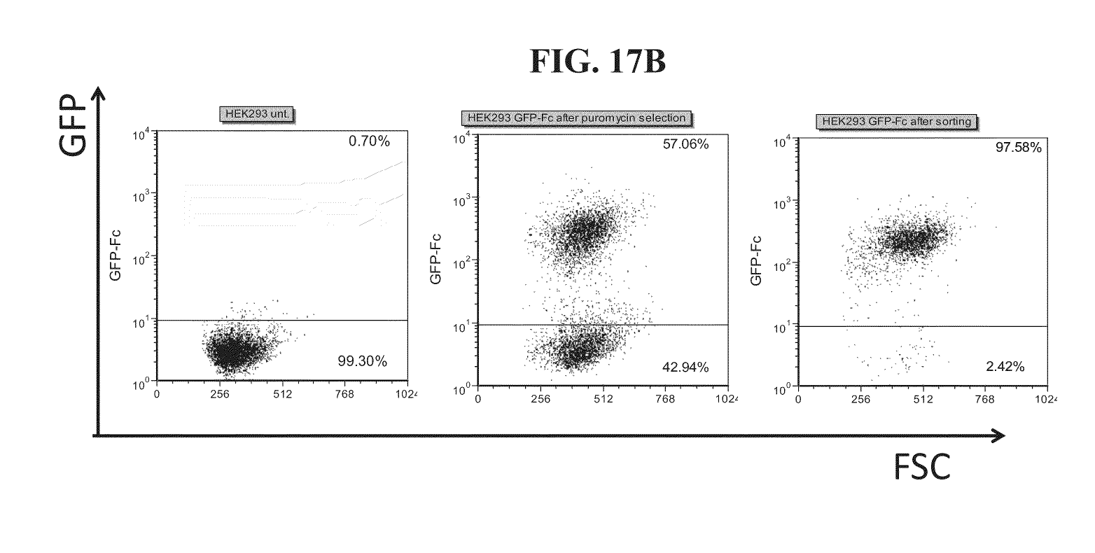

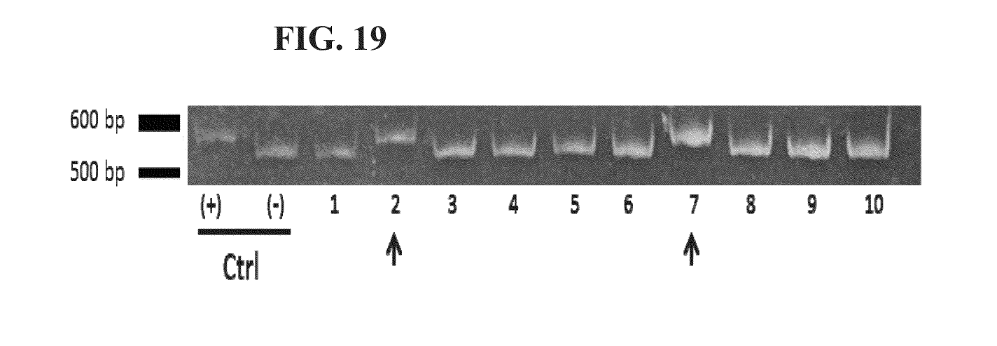

Exemplary expression systems including, CHO and HEK293 cells expressing a recombinant GFP-Fc protein (Example 2, FIGS. 17A-B and 18A-B) in which TSC expression was downregulated using CRISPR were generated demonstrating that deletion of TSC increases production and secretion of the recombinant GFP-Fc protein without compromising cell viability (Example 2, FIG. 19, 20A-B, 21-24).

Consequently, the present teachings suggest downregulating expression and/or activity of TSC for increasing production of a protein of interest in a cell.

Thus, according to a first aspect of the present invention, there is provided a method of expressing a polypeptide of interest.

The method is effected by providing a cell having been contacted with an agent which downregulates an expression of a tuberous sclerosis (TSC) protein or directly inhibits an activity of same; and contacting the cell with a polynucleotide encoding the recombinant polypeptide of interest.

Alternatively or additionally the method is effected by contacting a cell with:

(i) an agent which downregulates expression of a tuberous sclerosis (TSC) protein or directly inhibits an activity of same;

(ii) a polynucleotide encoding the recombinant polypeptide of interest.

According to an embodiment of the invention contacting (i) and contacting (ii) are performed concomitantly.

According to an embodiment of the invention contacting (i) and contacting (ii) are performed sequentially.

According to an embodiment of the invention contacting (i) is performed prior to contacting (ii).

According to an embodiment of the invention contacting (ii) is performed prior to contacting (i).

The methods described herein are aimed at improving production of recombinant proteins in host cell systems.

According to another aspect of the present invention, there is provided a method of increasing production of a recombinant polypeptide of interest, the method comprising:

providing a cell having been contacted with a polynucleotide encoding the recombinant polypeptide of interest; and

contacting the cell with an agent which downregulates an expression of a tuberous sclerosis (TSC) protein or directly inhibits an activity of same, thereby increasing production of the recombinant polypeptide of interest.

As used herein the term “production” refers to production using recombinant DNA techniques as is further described hereinbelow.

For the same culture conditions, the polypeptide production of the present invention is generally expressed in comparison to the polypeptide production in a cell of the same species expressing the polypeptide of interest but not contacted with the agent or contacted with a vehicle control, also referred to as control.

As used herein, the term “increased production” refers to an increase of at least 10% in the recombinant polypeptide production, as may be manifested in the amount of the polypeptide expressed in the cell and/or secreted into the medium, as compared to the control cell. According to a specific embodiment, the increase is in at least 10%, 20%, 30%, 40% or even higher say, 50%, 60%, 70%, 80%, 90% or more than 100%.

As used herein, the term “cell” refers to a eukaryotic cell which expresses TSC.

Methods of analyzing TSC expression are well known in the art e.g., PCR, Western-blot and flow cytometry and further described in the examples section which follows.

Examples of eukaryotic cells which may be used along with the teachings of the invention include but are not limited to, mammalian cells, fungal cells, yeast cells, insect cells, algal cells or plant cells.

According to a specific embodiment, the cell is a cell line.

According to another specific embodiment, the cell is a primary cell.

According to a specific embodiment the cell is grown in suspension.

According to a specific embodiment, the cell is an adherent cell grown in a monolayer.

According to specific embodiments the cell is approved by the FDA or other regulatory agency for use in recombinant protein production for clinical purposes.

According to specific embodiments the cell is a mammalian cell.

The cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells may be derived from any developmental stage including embryo, fetal and adult stages, as well as developmental origin i.e., ectodermal, mesodermal, and endodermal origin.

Non limiting examples of mammalian cells include monkey kidney CV1 line transformed by SV40 (COS, e.g. COS-7, ATCC CRL 1651); human embryonic kidney line (HEK293 or HEK293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); NIH3T3, Jurkat, canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC5 cells; FS4 cells; and a human hepatoma line (Hep G2), PER.C6, K562, and Chinese hamster ovary cells (CHO).

The CHO cells may include, but not be limited to, CHO/dhfr⁻ or CHO/DG44 cells. The Chinese hamster ovary tissue-derived CHO cell includes any cell which is a cell line established from an ovary tissue of Chinese hamster (Cricetulus griseus). Examples include CHO cells described in documents such as Journal of Experimental Medicine, 108, 945 (1958); Proc. Natl Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Natl Acad. Sci. USA, 77, 4216 (1980); Proc. Natl Acad. Sci., 60, 1275 (1968); Cell, 6, 121 (1975); Molecular Cell Genetics, Appendix I, II (pp. 883-900); and the like. In addition, CHO-K1 (ATCC CCL-61), DUXB11 (ATCC CCL-9096) and Pro-5 (ATCC CCL-1781) registered in ATCC (The American Type Culture Collection) and a commercially available CHO-S (Life Technologies, Cat #11619) or sub-cell lines obtained by adapting the cell lines using various media can also be exemplified.

According to a specific embodiment, the cell may be from a cell line used in hybridoma production. The term “hybridoma” refers to a hybrid cell line produced by the fusion of an immortal cell line of immunologic origin (e.g. myeloma) and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line which produces antibodies such as, for example, quadromas [See, e.g., Milstein et al., Nature, 537:3053 (1983)]. The hybrid cell line can be of any species, including human and mouse. Thus, the cell can be a myeloma cell, such as from murine myeloma lines, such as, but not limited to, MOPC-21, MPC-11, NSO, SP-2, Sp2/0, S 194, and X63-Ag8-653 cells; human myeloma cell lines, such as, but not limited to, Namalwa, Karpas 707H, RPMI 8226, 8226 AR/NIP4-1, KM-2R, and U-266; or rat myeloma cell lines, such as, but not limited to, YB2/0, YB2/3.0.Ag.20, Y3-Ag1.2.3, 112983F.

According to specific embodiments the mammalian cell is selected from the group consisting of a Chinese Hamster Ovary (CHO), HEK293, PER.C6, HT1080, NS0, Sp2/0, BHK, Namalwa, COS, HeLa and Vero cell.

According to other specific embodiments the mammalian cell is a Chinese Hamster Ovary (CHO) cell or a HEK293 cell.

According to a specific embodiment the cell may be independently modified to include mutations which simplify the cloning and selection of an expressing cell, and/or increase the secretion or expression of the polypeptide of interest. Such modifications may take place for example in the carbohydrate pathway, in glutamine synthetase (GS) and/or in dihydrofolate reductase (DHFR) (see e.g. Estes and Melville, Adv Biochem Eng Biotechnol (2014) 139: 11-33, the contents of which are incorporated herein by reference in their entirety).

According to specific embodiments the cell can be modified in one, two or all i.e. carbohydrate synthesis pathway, GS and DHFR.

According to specific embodiments modification the carbohydrate synthesis pathway refers to modification in the fucosylation pathway e.g. downregulation of FUT8. Dowregulation of the fucosylation pathway leading to reduced ability to fucosylate proteins may enhance protein effector functions. For example, Lack of fucosyl residues improves the binding antibodies to FcgRIIIa on macrophages and enhances ADCC (see e.g. U.S. Pat. No. 8,409,838 and U.S. Pat. No. 7,214,775).

The GS enzyme catalyzes the production of glutamine from glutamate and ammonia. A cell that lack GS (e.g. CHO-K1 of SAFC, can be obtained from e.g. Sigma) must be propagated in a medium containing glutamine unless the cell is stably transfected with a vector that expresses GS. Thus according to specific embodiments, modified in GS refers to dowregulation of GS. Methionine sulfoxamine (MSX) binds to the GS enzyme thereby prevents the production of glutamine. A cell modified to upregulate GS can survive higher levels of MSX. Thus, according to other specific embodiments, modified in GS refers to upregulation of GS.

DHFR catalyzes the reduction of 5, 6-dihydrofolate to 5, 6, 7, 8-tetrahydrofolate, which is essential for DNA synthesis. A cell that lack DHFR (e.g. CHO-derived DG44 cells, can be obtained from e.g. Life Technologies, CHO DHFR−/−, can be obtained from e.g. Sigma) must be propagated in medium containing the purine precursors hypoxanthine and thymidine (HT) unless the cell is stably transfected with a vector that expresses DHFR. Thus, according to specific embodiments, modified in DHFR refers to dowregulation of DHFR. Methotrexate (MTX), a drug analog to folate, binds to DHFR, thereby inhibiting the production of tetrahydrofolate. Upon MTX treatment cells expressing insufficient levels of DHFR are deprived of nucleoside precursors and die. A cell modified to upregulate DHFR can survive higher levels of MTX. Thus, according to other specific embodiments, modified in DHFR refers to upregulation of DHFR.

The terms “polypeptide” and “protein” are interchangeably used. As used herein, the term “recombinant polypeptide” refers a polypeptide produced by recombinant DNA techniques, i.e., produced from cells transformed by an exogenous DNA construct encoding the polypeptide. The recombinant polypeptide can be foreign to the cell (i.e. a human polypeptide expressed in a CHO cell) or a homologous polypeptide derived from a nucleic acid sequence not from its natural location and expression level in the genome of the cell.

The term “amino acid” is understood to include the 20 naturally occurring amino acids; those amino acids often modified post-translationaly in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The polypeptide can be long e.g., more than 50 amino acids or short e.g., 2-50 amino acids long.

The polypeptide can be a naturally occurring or a synthetic polypeptide, e.g., chimeric polypeptide e.g., Enbrel, CTLA-Ig or factor VIII-Fc.

The polypeptide may refer to a single molecule or a complex of a two or more polypeptide chains which are non-covalently or covalently (e.g., antibodies) assembled.

The polypeptide may be intracellulary expressed or secreted to the culture medium.

The polypeptide may include additional amino acid sequences which can facilitate the purification process (e.g., affinity tags e.g. GST protein, FLAG peptide, or His-tag).

The recombinant polypeptide of interest is any type of recombinant polypeptide having commercial value that can be used in medicine, diagnostics, agriculture and biotechnology processes. Non-limiting examples of such polypeptides include hormones, cytokines, receptors, soluble receptors, interleukins, growth factors, antibodies, Specific examples include, but are not limited to, growth hormone, including human growth hormone; bovine growth hormone; growth hormone releasing factor; parathyroid hormone; thyroid stimulating hormone; lipoproteins; alpha-1-antitrypsin; caerulein; motilin; bombesin; neurotensin; bradykinin; substance P; analgesic substances like enkephalin, endorphin, daynorphin and kyotorphin; insulin A-chain; insulin B-chain; proinsulin; follicle stimulating hormone; somatostatin; prolactin; rennin; vasopressin; oxytocin; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES; human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; gastrin; secretin; pancreozymin; cholecystokinin; angiotensin; human placenta lactogen; human chorionic gonadotropin (HCG); a microbial protein; such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon (IFN) such as IFN-alpha, -beta, and -gamma; colony stimulating factors (CSFs), e.g., M-CSF, GM-CSF, and G-CSF; interleukins (ILs), e.g., IL-1 to IL-10; interferons such as interferon-alpha,-beta and gamma; superoxide dismutase; T-cell receptors; surface membrane proteins; decay accelerating factor; viral antigen such as, for example, a portion of the AIDS envelope; transport proteins; homing receptors; addressins; regulatory proteins; antibodies; chimeric proteins; and fragments of any of the above-listed polypeptides.

According to specific embodiments, the recombinant polypeptide is a human recombinant polypeptide.

According to specific embodiments the polypeptide is a secreted polypeptide.

According to specific embodiments the polypeptide comprises an antibody or an antibody fragment.

The term “antibody” as used in this invention includes intact molecules as well as functional fragments thereof, such as Fc fusion proteins, Fab, F(ab′)2, and Fv that are capable of binding to macrophages. The term refers to any antibody subtype including IgG (e.g., IgG1, IgG4), IgA and IgM.

The antibody can be a primary antibody that targets directly a target of interest or a secondary antibody that targets a primary antibody.

The antibody can be a monospecific antibody (i.e. binds one antigen) or bispecific (i.e binds two different antigens).

The antibody can be a human antibody or a humanized antibody. Humanized forms of non-human (e.g., murine) antibodies are chimeric molecules of immunoglobulins, immunoglobulin chains or fragments thereof which contain minimal sequence derived from non-human immunoglobulin. Methods for producing human antibodies or for humanizing non-human antibodies are well known in the art, see for example Jones et al., Nature, 321:522-525 (1986); Riechmann et al., Nature 332:323-327 (1988); Verhoeyen et al., Science, 239:1534-1536 (1988) and U.S. Pat. No. 4,816,567; Hoogenboom and Winter, J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boerner et al., J. Immunol., 147(1):86-95 (1991); U.S. Pat. Nos. 5,545,807; 5,545,806; 5,569,825; 5,625,126; 5,633,425; 5,661,016, Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

The light and heavy chains of the antibody may be encoded in the same plasmid or in two separate plasmids in the same or in different cells.

According to a specific embodiment, the light and heavy chains may be transformed into separate modified host cell cultures, either of the same or of differing species.

According to specific embodiments either one or both cell cultures are modified to dowregulate expression or activity of a TSC protein.

According to another specific embodiment, separate plasmids for the light and heavy chains may be used to co-transform a single modified host cell culture.

According to another specific embodiment, a single expression plasmid containing both genes and capable of expressing the genes for both light and heavy chains may be transformed into a single modified host cell culture.

When heavy and light chains are co-expressed in the same host, the isolation procedure is designed so as to recover reconstituted antibody. This can be accomplished by conventional antibody purification procedures such as, for example, protein A-Sepharose, hydroxylapatite chromatography, gel electrophoresis, dialysis, or affinity chromatography.

According to specific embodiments the antibody or antibody fragment is a probody.

As used herein, the term “probody” refers to a proteolitically activated antibody or fragment thereof which includes a masking peptide linked to the N-terminus of the light chain of the antibody through a protease-cleavable linker peptide. In the intact form, the probody is effectively blocked from binding to the target antigen; however, once activated by appropriate proteases (e.g. in diseased environment), the masking peptide is released, revealing a fully active antibody capable of binding to its target. Typically, probodies are engineered to remain inert until activated locally in diseased tissue wherein protease activity is upregulated, e.g. inflammatory conditions, e.g. cancer [see e.g. Pulo and Lowman, Expert Opin Biol Ther (2014) 14(8): 1049-53].

Exemplary antibodies produced in the cells of the present invention include, but are not limited to, abciximab (ReoPro®), adalimumab (Humira®), alemtuzumab (Campath®), basiliximab (Simulect®), bevacizumab (Avastin®), cetuximab (Erbitux®), daclizumab (Zenapax®), dacetuzumab, eculizumab (Soliris®), efalizumab (Raptiva®), Edrecolomab (Panorex®), epratuzumab, ibritumomab (Zevalin®), tiuxetan, infliximab (Remicade®), muromonab-CD3 (OKT3), natalizumab (Tysabri®), omalizumab (Xolair®), palivizumab (Synagis®), panitumumab (Vectibix®), ranibizumab (Lucentis®), gemtuzumab ozogamicin (Mylotarg®), oregovomab (OvaRex®), rituximab (Rituxan®), tositumomab (Bexxar®), trastuzumab (Herceptin®), MetMAb, ocrelizumab, pertuzumab, Raptiva® (efalizumab), hu M195Mab, MDX-210, BEC2, anti-Abeta, anti-CD4, anti-IL-13, anti-oxLDL, trastuzumab-DM1, apomab, rhuMAb beta7, rhuMAb IFNalpha, GA101, anti-OX40L, ipilimumab, Valortim, ustekinumab, golimumab, ofatumumab, zalutumumab, tremelimumab, motavizumab, mitumomab, ecromeximab, ABX-EGF, MDX010, XTL 002, H11 SCFV, 4B5, XTL001, MDX-070, TNX-901, IDEC-114, and any antibody fragments specific for antigens including but not limited to complement C5, CBL, CD147, gp 120, VLA4, CD11a, CD18, VEGF, CD40L, anti-Id, ICAM1, CD2, EGFR, TGF-beta2, TNF-alpha, TNF receptor, E-selectin, FactII, Her2/neu, F gp, CD11/18, CD14, CD80, ICAM3, CD4, CD23, beta.2-integrin, alpha4beta7, CD52, CD22, OX40L, IL-5 receptor, GM-CSF receptor, GM-CSF, HLA-DR, oxLDL, CD64 (FcR), TCR alpha beta, CD3, Hep B, CD125, DR5, EpCAM, gpIIbIIIa, IgE, beta 7 integrin, CD20, IL1beta, IL-2, IL-4, IL-5, IL-6, IL-8, IL-9, IL10, IL13, IL-12/IL-23, IL-1 5, IFN-alpha, IFN-beta, IFN-gamma, VEGFR-1, platelet-derived growth factor receptor .alpha. (PDGFRalpha), vascular adhesion protein 1 (VAP1), connective tissue growth factor (CTGF), Apo2/TRAIL, CD25, CD33, HLA, F gp, IgE, CTLA-4, IP-10, anti-C. difficile Toxin A and Toxin B, B. anthracis PA, respiratory syncytial virus (RSV), mannose receptor/hCG.beta, integrin receptors, PD1, PDL-1, CD19, CD70, and VNR integrin.

According to specific embodiments the polypeptide is selected from the group consisting of CTLA4-Ig, IFNβ, IFNγ, TNFα and IL-6.

It is expected that during the life of a patent maturing from this application many relevant recombinant polypeptides will be developed and the scope of the term recombinant polypeptide is intended to include all such new technologies a priori.

As mentioned, the cell is contacted with a polynucleotide encoding a recombinant polypeptide thereby modified to express the recombinant polypeptide.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The polynucleotide encoding the polypeptide of interest can be introduced into the cell using methods which are well known in the art and further described hereinbelow with respect to an agent capable of upregulating expression of an anti-apoptotic gene.

As mentioned, the method of this aspect of the present invention is effected by providing a cell having been contacted with an agent which downregulates an expression of a tuberous sclerosis (TSC) protein or directly inhibits an activity of same or by contacting the cell with an agent which downregulates expression of a tuberous sclerosis (TSC) protein or directly inhibits an activity of same.

As used herein the phrase “tuberous sclerosis (TSC) protein” encompasses TSC1 protein, TSC2 protein and the heterodimeric protein complex formed by TSC1 and TSC2 (denoted herein as TSC1/TSC2 complex). As used herein, TSC protein refers to functional TSC and fragments thereof able to inhibit mTOR activation, and more specifically mTORC1.

According to specific embodiments TSC is TSC1.

TSC1 is also known as hamartin. According to specific embodiments, the TSC1 protein refers to the Chinese hamster protein, such as provided in the following GenBank Numbers XP_007651756 (SEQ ID NO: 1), XP_007651757 (SEQ ID NO: 2) and XP_007614219 (SEQ ID NO: 3). According to other specific embodiments, the TSC1 protein refers to the murine protein such as provided in the following GenBank Number NP_075025 (SEQ ID NO: 4). According to a specific embodiment, the TSC1 protein refers to the human protein, such as provided in the following GenBank Numbers NP_000359 (SEQ ID NO: 5), NP_001155898 (SEQ ID NO: 6), and NP_001155899 (SEQ ID NO: 7).

As used herein “TSC1 protein” refers to a functional TSC1 and fragments thereof able to form a complex with TSC2 and inhibit mTOR activation, specifically mTORC1.

According to specific embodiments TSC is TSC2.

TSC2 is also known as tuberin. According to specific embodiments, the TSC2 protein refers to the murine protein, such as provided in the following GenBank Numbers NP_035777 (SEQ ID NO: 8), NP_001034452 (SEQ ID NO: 9), NP_001273642 (SEQ ID NO: 10), NP_001273647 (SEQ ID NO: 11) and NP_001273649 (SEQ ID NO: 12). According to other specific embodiments, the TSC2 protein refers to the Chinese hamster protein such as provided in the following GenBank Numbers XP_007640393 (SEQ ID NO: 13), XP_003501554 (SEQ ID NO: 14) and XP_007606632 (SEQ ID NO: 15). According to a specific embodiment, the TSC2 protein refers to the human protein, such as provided in the following GenBank Number NP_000539 (SEQ ID NO: 16), NP_001070651 (SEQ ID NO: 17), and NP_001107854 (SEQ ID NO: 18). TSC2 contains a GTPase activating protein (GAP) domain which has been shown to stimulate the GTPase activity of the small GTPase Rheb protein, which in its GTP bound form is an activator of mTORC1.

As used herein “TSC2 protein” refers to functional TSC2 and fragments thereof able to stimulate the GTPase activity of the Rheb protein and/or able to form a complex with TSC1, thereby inhibiting mTOR activation, specifically mTORC1.

The terms “TSC1” and “TSC2” also refers to functional TSC1 and TSC2 homologues which exhibit the desired activity (i.e., inhibiting activation of mTOR). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptides set forth in SEQ ID NOs: 1-18, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow). The homolog may also refer to an ortholog, a deletion, insertion, or substitution variant, including an amino acid substitution.

Sequence identity or homology can be determined using any protein or nucleic acid sequence alignment algorithm such as Blast, ClustalW, MUSCLE, and HHpred.

As used herein the phrase “dowregulates expression” refers to dowregulating the expression of a protein (e.g. TSC) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not contacted with the agent or contacted with a vehicle control, also referred to as control.

Down regulation of protein expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

As used herein, the phrase “directly inhibits activity” refers to the ability to directly decrease the intrinsic catalytic activity of a protein, inhibit the interaction of a protein with its target proteins and/or inhibit the formation of a complex containing the protein (e.g. TSC1/TSC2 complex).

For the same culture conditions the activity is generally expressed in comparison to the activity in a in a cell of the same species but not contacted with the agent or contacted with a vehicle control, also referred to as control.

Inhibiting activity of a protein may be either transient or permanent.

According to specific embodiments, inhibiting activity refers to completely inactive protein, as detected by immunoprecipitation or an enzyme activity assay such as in-situ activity assay or in-vitro activity assay.

According to other specific embodiments inhibiting activity refers to an observable decrease in the activity of the protein. The reduction may be by at least a 10%, at least 20%, at least 30%, at least 40%, at least 50%, at least 60%, at least 70%, at least 80%, at least 90%, at least 95% or at least 99% reduction.

The expression level and/or activity level of TSC expressed in the cells of some embodiments of the invention can be determined using methods known in the arts and further described hereinbelow.

Down regulation of the TSC protein can be at the protein or nucleic acid level (i.e. DNA or RNA) affecting the expression levels or the activity of the TSC protein. Non-limiting examples of agents capable of down regulating TSC activity or expression are described in details hereinbelow.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, downregulation of TSC can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., TSC1 and/or TSC2) and does not cross inhibit or silence other targets as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the TSC mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, suitable siRNAs directed against the human TSC2 can be the SignalSilence® Tuberin/TSC2 siRNA from Cell Signaling Technology (cat. no. 6476), Tuberin siRNA (h) sc-36762 from Santa Cruz, and TSC2 FlexiTube siRNA from Qiagen cat. no. SI00011697.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence 5′-AAC ACC AAG ATA CCT GCT TGG GTC-3′ (SEQ ID NO: 19) or variants thereof.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a TSC can be effected using an antisense polynucleotide capable of specifically hybridizing with an mRNA transcript encoding TSC.

Design of antisense molecules which can be used to efficiently downregulate a TSC must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

For example, suitable antisense oligonucleotides targeted against the TSC1 mRNA (which is coding for the TSC1 protein) would be of the following sequences: accacctaca cacccaccca (SEQ ID NO: 37) for Chinese hamster TSC1; gcctcctccc acctcttagt (SEQ ID NO: 38) for human TSC1; and catcccactc tctgccctct (SEQ ID NO: 39) for mouse TSC1.

For example, suitable antisense oligonucleotides targeted against the TSC2 mRNA (which is coding for the TSC2 protein) would be of the following sequences: atccctctcc accctcttgc (SEQ ID NO: 40) for Chinese hamster TSC2; gtccctctct actctcttgc c (SEQ ID NO: 41) for human TSC2; and tccctttcta ccctctttcc c (SEQ ID NO: 42) for mouse TSC2.

Nucleic acid agents can also operate at the DNA level as summarized infra.

Downregulation of TSC can also be achieved by inactivating the gene (e.g., TSC1 and/or TSC2) via introducing targeted mutations involving loss-of function alterations (e.g. point mutations, deletions and insertions) in the gene structure.

As used herein, the phrase “loss-of-function alterations” refers to any mutation in the DNA sequence of a gene (e.g., TSC1 and/or TSC2) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments los-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. TSC may be in a homozygous form or in a heterozygous form. According to this embodiment, homozygosity is a condition where both alleles at the e.g. TSC locus are characterized by the same nucleotide sequence. Heterozygosity refers to different conditions of the gene at the e.g. TSC locus.

Methods of introducing nucleic acid alterations to a gene of interest are well known in the art [see for example Menke D. Genesis (2013) 51: −618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and nonhomologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—

Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www.talendesign.org). TALEN can also be designed and obtained commercially from e.g., Sangamo Biosciences™ (Richmond, Calif.).

CRISPR-Cas System—

Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH—, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present invention include 5′-GTGCAATACCGGTTGAGAATTGG-3′ (SEQ ID NO: 20) which correspond to exon 2 of CHO TSC1 gene, 5′-GCAGATGGACACCGACGTTGTGG-3′ (SEQ ID NO: 21) which correspond to exon 4 of CHO TSC1 gene; 5′-ATGACAAGCACCTCTTGGAC-3′ (SEQ ID NO: 22) which correspond to exon 4 of human TSC1 gene, 5′-CTACCAATGATTCCACAGTC-3′ (SEQ ID NO: 23) which correspond to exon 6 of human TSC1 gene; 5′-GTCTTTAGGGTGACCGTTTGGGG-3′ (SEQ ID NO: 24) which correspond to exon 4 of CHO TSC2 gene, 5′-tcttcgtagggatggcactc-3′ (SEQ ID NO: 25), which targets exon 10 of CHO TSC2 gene, 5′-GAGAGCCATGGAACTCGTTCTGG-3′ (SEQ ID NO: 26) which correspond to exon 11 of CHO TSC2 gene; 5′-CGAAGACCTTCACGAAAGGC-3′ (SEQ ID NO: 27) which correspond to exon 6 of human TSC2 gene and 5′-AACAATCGCATCCGGATGAT-3′ (SEQ ID NO: 28) which correspond to exon 3 of human TSC2 gene.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas To12 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of TSC may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Down-Regulation at the Polypeptide Level

According to specific embodiments the agent capable of downregulating a TSC is an antibody or antibody fragment capable of specifically binding TSC. Preferably, the antibody specifically binds at least one epitope of a TSC. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

As TSC is localized intracellularly, an antibody or antibody fragment capable of specifically binding TSC is typically an intracellular antibody. Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Another agent which can be used along with some embodiments of the invention to downregulate TSC is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent capable of downregulating TSC would be any molecule which binds to and/or cleaves TSC. Such molecules can be a small molecule, TSC antagonists, or TSC inhibitory peptide.

Alternatively or additionally, small molecule or peptides can be used which interfere with TSC protein function (e.g., catalytic or interaction).

According to specific embodiments the agent interferes with the formation of a TSC1/TSC2 complex.

According to other specific embodiments the agent interferes with TSC1/TSC2 complex interaction with its binding partners (e.g., competitive inhibitor).

Determining the extent of binding to a TSC can be effected using techniques standard in the art including, but not limited to, immunoprecipitation, mass spectrometry and gel filtration assays.

It will be appreciated that a non-functional analogue of at least a catalytic or binding portion of TSC can be also used as an agent which downregulates TSC.

According to specific embodiments, the invention further contemplates an agent for dowregulating apoptosis. As used herein, the term “apoptosis” is intended to cover all forms of programmed cell death.

Down regulating apoptosis can be effected for example by dowregulating expression or activity of a pro-apoptotic protein or by upregulating expression or activity of an anti-apoptotic protein.

According to specific embodiments the method further comprising contacting the cell with an agent which downregulates an activity and/or expression of a pro-apoptotic gene.

According to other embodiments the article of manufacture further comprising an agent for down regulating an activity and/or expression of a pro-apoptotic gene.

According to other specific embodiments the isolated cell further comprising an exogenous agent which downregulates an activity and/or expression of a pro-apoptotic gene.

As used herein, the term “pro-apoptotic gene” refers to a gene that promotes apoptotic cell death. Non limiting examples of pro-apopatotic gene that can be used in the present inventions includes: BAX, BAK, BOK, Bod, Bcl-XS, Bcl-G, BID, Bim, Bid, Bad, Bmf, Bim, Blk, Nbk, Diva, Hrk, Nix, Bnip3, Bnip3L, Noxa, PUMA, Egl-1, Bcl-rambo, SMAC, PTEN, Fas, FasL, FADD, TRAIL, TNF-R1, TNFRSF10A, TNFRSF10B, TNFRSF10C, TNFRSF10D, TNFRSF11B, FADD, Caspase 1, Caspase 2, Caspase 3, Caspase 4, Caspase 5, Caspase 6, Caspase 7, Caspase 8, Caspase 9, Caspase 10, Caspase 12, Caspase 14, APAF1, HTRA2, KEAP1, SHC1, ZNHIT1, LGALS3, HI95, p53AEP1, TGF-β, Granzyme A and Granzyme B.

According to specific embodiments is selected from the group consisting of BAX, BAK and PUMA.

Down regulation of a pro-apoptotic gene can be effected on the genomic, the transcript or the protein level as further disclosed hereinabove for TSC.

According to other embodiments the method further comprising contacting the cell with an agent which upregulates an activity and/or expression of an anti-apoptotic gene.

According to other embodiments the article of manufacture further comprising an agent for up regulating an activity and/or expression of an anti-apoptotic gene.

According to other specific embodiments the isolated cell further comprising an exogenous agent which upregulates an activity and/or expression of an anti-apoptotic gene.

As used herein, the term “anti-apoptotic gene” refers to a gene that inhibits apoptotic cell death. Non limiting examples of anti-apoptotic genes that can be used in the present inventions include: Bcl-2, Mcl-1, Bcl-X, Bcl-x1, Bcl-w, BFL1, A1, Bcl-B, BOO/DIVA, A1/Bfl-1, NR—B, Bcl2-L-10, p35, FLIP, BIRC1, CIAP1, CIAP2, BIRC4, Survivin, APOLLON and LIVIN.

According to specific embodiments the anti-apoptotic gene is selected from the group consisting of Bcl-2, Bcl-xL, Bcl-w and Mcl-1 and XIAP.

Upregulation of a gene can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like).

For the same culture conditions the expression is generally expressed in comparison to the expression in a cell of the same species but not contacted with the agent or contacted with a vehicle control, also referred to as control.

Upregulation of protein expression and/or activity may be either transient or permanent.

According specific embodiments up regulating an activity and/or expression refers to an increase in the activity as detected by or an enzyme activity assay such as in-situ activity assay or in-vitro activity assay and/or expression of the protein as detected by RT-PCR or Western blot. The increase may be by at least a 10%, at least 20%, at least 30%, at least 40%, at least 50%, at least 60%, at least 70%, at least 80%, at least 90%, at least 95%, at least 100% or more.

The expression level and/or activity level of an anti-apoptotic gene expressed in the cells of some embodiments of the invention can be determined using methods known in the arts, e.g but not limited to selectable marker gene, Northern blot analysis, PCR analysis, Western blot analysis, Enzyme linked immunosorbent assay (ELISA), RNA in situ hybridization stain, In situ RT-PCR stain, Immunohistochemistry, Radio-immunoassay (RIA), Fluorescence activated cell sorting (FACS, also referred as flow cytometry), In situ activity assay, In vitro activity assay.

Following is a non-limiting list of agents capable of upregulating the expression level and/or activity of a gene giving an anti-apoptotic gene as an example, that can be used according to specific embodiments of the present invention.

An agent capable of upregulating expression of an anti-apoptotic gene may be an exogenous polynucleotide sequence designed and constructed to express at least a functional portion of the anti-apoptotic gene. Accordingly, the exogenous polynucleotide sequence may be a DNA or RNA sequence encoding an anti-apoptotic molecule, capable of increasing cell viability.

Methods for expressing a recombinant polypeptide in a cell are well known in the art [see e.g. Goeddel et al., Methods Enzymol. 185 (1990) 3-7; Wurm and Bernard, Curr. Opin. Biotechnol. 10 (1999) 156-159] and are further described hereinbelow.

To express an exogenous polypeptide in a cell (e.g. eukaryotic cell, e.g. mammalian cell), a polynucleotide sequence encoding the polypeptide is preferably ligated into a nucleic acid construct suitable for cell expression. Such a nucleic acid construct includes regulatory sequences that direct constitutive expression of a nucleotide sequence as well as those that direct inducible expression of the nucleotide sequence only under certain conditions.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed.

The promoter may be inducible or constitutive.

Non-limiting examples of suitable promoters for use in eukaryotic host cells include, a CMV immediate early promoter, an HSV thymidine kinase promoter, an early or late SV40 promoter, LTRs from retroviruses, a mouse metallothionein-I promoter and the tetracycline-inducible promoter.

According to some embodiments, the agent for dowregulating expression of activity of a TSC and optionally an agent for downregulating apoptosis are provided in a formulation suitable for cell penetration that enhances intracellular delivery of the agent.

Any suitable penetrating agent for enhancing penetration of the agent to a cell may be used, as known by those of skill in the art.

Thus, according to specific embodiments the agent provided herein can be functionally associated with a cell-penetrating peptide. As used herein, a “cell-penetrating peptide (CPP)” is a peptide that comprises a short peptides ((≦40 amino acids) or functional motif that confers the energy-independent (i.e., non-endocytotic) translocation properties associated with transport of the membrane-permeable complex across the plasma and/or nuclear membranes of a cell. They have the exceptional property of carrying into the cells a wide variety of covalently and noncovalently conjugated cargoes such as proteins, oligonucleotides, and even 200 nm liposomes.

The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP.

Protocols for producing CPPs-cargos conjugates and for infecting cells with such conjugates can be found, for example in L. Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633].

According to specific embodiments, the agent of the present invention is attached to a functional moiety such as a detectable moiety that allows detection of the cell population that contains the agent.

Thus, the agent may be attached to a detectable moiety, including but not limited to a fluorescent moiety, radioactive moiety, a chemiluminescent moiety, an affinity moiety, an enzyme, or a magnetic moiety.

The detectable moiety may be conjugated translationally to the agent by fusing the nucleic acid sequence encoding the detectable moiety to the agent.

Alternatively, the detectable moiety may be attached to the agent by chemical conjugation using any conjugation method known to one skilled in the art.

Alternatively, the agent may be attached to an affinity moiety capable of selective interaction with a cognate binding moiety, such as for example biotin/avidin, ligand/receptor, and the like.

Various methods, widely practiced in the art, may be employed to attach the above described moieties to the agent disclosed by the invention. Functional moieties, such as fluorophores, biotin and streptavidin are commercially available from essentially all major suppliers of e.g. immunofluorescence flow cytometry reagents (for example, Pharmingen or Becton-Dickinson).

It will be appreciated that if the agent is attached to a fluorescent moiety (either directly, or indirectly through a cognate binding molecule), the modified cell population (i.e., in which activity or expression of TSC has been down-regulated) may be selected using known cell sorting procedures such as by using a fluorescence-activated cell sorter (FACS).

A multitude of flow cytometers are commercially available including for e.g. Becton Dickinson FACScan and FACScaliber (BD Biosciences, Mountain View, Calif.). Antibodies that may be used for FACS analysis are taught in Schlossman S, Boumell L, et al, [Leucocyte Typing V. New York: Oxford University Press; 1995] and are widely commercially available.

If the agent is attached to a magnetic moiety (either directly, or indirectly through a cognate binding molecule), the modified cell population may be selected by magnetic activated cell separation.

If the agent is attached to an affinity moiety, the modified cell population may be depleted of non-modified cells by affinity purification with the cognate binding molecule. Thus, for example, if the fucose binding agent is attached to biotin, the mutated cell population may be depleted of unwanted cells by purification with strepavidin beads or column. If, for example the fucose binding agent is attached to an antibody or an Fc of an antibody, the mutated cell population may be depleted of unwanted cells by purification with protein A beads or column.

According to another aspect of the present invention, there is provided a method of selecting an agent which downregulates expression of a tuberous sclerosis (TSC) protein or directly inhibits an activity of same, the method comprising:

(a) contacting a population of cells expressing a reporter polypeptide with an agent putative for down regulating expression of a TSC protein or directly inhibiting an activity of same; and

(b) determining whether expression and/or secretion of said polypeptide increases following said contacting with said agent;

wherein an increase above a predetermined threshold indicates said agent downregulates expression of a TSC protein or directly inhibits an activity of same.

As used herein the term “reporter polypeptide” refers to a polypeptide which specific expression can be qualified and preferably quantified

As used herein the phrase “an increase above a predetermined threshold” refers to an increase in expression and/or secretion of said polypeptide which is higher than a predetermined threshold such as at least 10%, at least 20%, at least 30%, at least 40%, at least 50%, at least 60%, at least 70%, at least 80%, at least 90%, at least 95%, at least 100% or more, relative to the polypeptide expression and/or secretion in a control cell that was not contacted with the agent or contacted with a vehicle control, also referred to as control.

Determining the expression and/or secretion of the polypeptide can be effected by any known method known the art, such as, but not limited to, PCR, ELISA, pulse-chase analysis, western blot, flow cytometry and immunohistochemistry.

According to specific embodiments the determining is effected by flow cytometry, western blot and/or ELISA.

Typically, an agent which dowregulates expression of a TSC or inhibits activity of same induces mTOR activation.

According to specific embodiments, the agent which downregulates expression of a TSC protein or directly inhibits an activity of same increases phosphorylation of S6.

According to other specific embodiments, the agent which downregulates expression of a TSC protein or directly inhibits an activity of same does not significantly affect autophagy of the cell.

According to specific embodiments, the agent which downregulates expression of a TSC protein or directly inhibits an activity of same does not affect cell proliferation in a statistically significant manner.

According to other specific embodiments the agent which downregulates expression of a TSC protein or directly inhibits an activity of same does not affect cell viability in a statistically significant manner.

Contacting cells with the agent and/or the polynucleotide encoding the recombinant polypeptide can be performed by in-vitro conditions including for example, adding the agent to the cells such that the agent is in direct contact with the cells, transfection, electroporation and infection with recombinant bacterial or viral vectors. The conditions used for contacting the cells are selected to induce efficient cellular changes, such as changes in transcription and/or translation rate and the like.

According to specific embodiments the contacting is effected ex-vivo or in-vitro.

Thus, regardless of the method of introduction, the present teachings provide for an isolated cell which comprises a recombinant polypeptide and an agent which downregulates expression of a TSC protein or directly inhibits an activity of same, as described herein.

According to another embodiment the isolated cell is obtainable according to any of the methods described herein.

Following contacting with the agent the degree of dowregulation of TSC, the integration site and/or the sequence alteration can vary between the transfected cells. Thus, the isolated cell may comprise a homogenous or heterogeneous population of cells. According to specific embodiments, the isolated cell comprises a heterogeneous population of cells.

As used herein, the term “heterogeneous” refers to less than 90% of the cells exhibit a specific trait of interest e.g. no expression of TSC as determined by e.g. RT-PCR.

According to other specific embodiments, the isolated cell comprises a homogenous population of cells.

As used herein, the term “homogenous” refers to more than 90% of the cells exhibit a specific trait of interest e.g. no expression of TSC as determined by e.g. RT-PCR.

It is appreciated that the degree of dowregulation of expression or activity of a TSC protein is correlated with the amount of the recombinant polypeptide production.

Thus, According to specific embodiments, following contacting a population of cells expressing a recombinant protein with an agent which downregulates expression or activity of a TSC protein, a cell containing the agent is selected and isolated.

According to specific embodiments the cell exhibiting the most significant reduction in TSC expression or activity is selected.

According to other specific embodiments the cell exhibiting the highest yield of recombinant protein production per fixed volume of culture is selected.

According to specific embodiments following contacting the cell comprising the agent is selected.

It will be appreciated that selection of the cell may be effected in a number of rounds (e.g. two, three or more rounds) of sequential selection. Further, the selection steps may comprise a number of rounds of sequential selection using the same method (e.g. solely FACS based separation or antibiotic resistance) or may combine a number of different methods (e.g. antibiotic resistance, followed by fluorescence based separation).

According to one embodiment, the number of rounds of selection and the specific method is selected such that cells which do not contain the agent are substantially removed.

The term “substantially removed” is intended to mean removal of at least 50% or more of the particular cell type, such as at least about 75%, about 80%, about 90%, about 95%, or about 97%, including at least 99%, 99.5%, 99.9% or more of the particular cell type.

Following isolation of the cell of the present invention, it may be grown in cultures, and in any apparatus that may be used to grow cultures, including fermentors or bioreactors. They may be grown as monolayers or attached to a surface. Alternatively, the isolated cell populations may be grown in suspension.

According to specific embodiments there is provided a cell culture comprising the isolated cell of the present invention and a cell culture medium.

As used herein, the term “cell culture” refers to a cell population that is grown under controlled conditions outside of its natural environment (i.e. in-vitro or ex-vivo). Typically, the cell population is grown with appropriate defined culture medium containing nutrients that nourish the cell which support its survival and optimally fertilization.

Cell culture procedures for both large and small-scale production of recombinant polypeptides are encompassed by the present invention. The procedures include, but not limited to, a fluidized bed bioreactor, shaker flask culture, or stirred tank bioreactor system operated e.g. in a batch, split-batch, fed-batch, or perfusion mode.

Following expression, the recombinant polypeptide is recovered by collecting the whole fermentation medium containing the polypeptide and/or the cells containing the polypeptide.

According to specific embodiments the method further comprising isolating said recombinant polypeptide.

As used herein, the term “isolated” refers to at least partially separated from the natural environment e.g., a cell.

The term “isolated” does not exclude the presence of the same polypeptide in alternative physical forms, such as dimers or alternatively glycosylated or derivatized forms.

According to specific embodiments, the isolated recombinant polypeptide is essentially free from contaminating cellular components such as carbohydrate, lipid or other impurities.

Methods for isolation and purification of polypeptides are well known in the art, see for example Chromatography, 5^{th} edition, Part A: Fundamentals and Techniques, Heftmann, E. (ed), Elsevier Science Publishing Company, New York, (1992); Advanced Chromatographic and Electromigration Methods in Biosciences, Deyl, Z. (ed.), Elsevier Science B V, Amsterdam, The Netherlands, (1998); Chromatography Today, Poole, C. F., and Poole, S. K., Elsevier Science Publishing Company, New York, (1991); Scopes, Protein Purification: Principles and Practice (1982); Sambrook, J., et al. (ed), Molecular Cloning: A Laboratory Manual, Second Edition, Cold Spring Harbor Laboratory Press, Cold Spring Harbor, N.Y., 1989; or Current Protocols in Molecular Biology, Ausubel, F. M., et al. (eds), John Wiley & Sons, Inc., New York.

According to specific embodiments, at least 80%, at least 90%, at least 95% or at least 99% of the total protein in the preparation is the recombinant polypeptide of interest.

According to specific embodiments, the isolated recombinant polypeptide is purified to a pharmaceutically acceptable purity.

Methods for evaluating protein purity are well known in the art and include SEC-HPLC, peptide mapping, SDS gel analysis and ELISA for specific contaminants.

According to another aspect there is provided an article of manufacture or a kit identified for recombinant expression of a recombinant polypeptide of interest comprising a packaging material packaging an agent for down regulating expression of a tuberous sclerosis (TSC) protein or directly inhibiting an activity of same; and a nucleic acid construct for expressing the polypeptide of interest.

The agent and the nucleic acid construct can be packaged in separate containers of in c-formulation. Methods for generating a nucleic acid construct for expressing a polypeptide of interest were described in details hereinabove.

According to another aspect there is provided an article of manufacture or a kit identified for recombinant expression of a recombinant polypeptide of interest comprising a packaging material packaging an isolated cell having been contacted with an agent which downregulates an expression of a tuberous sclerosis (TSC) protein or directly inhibits an activity of same, wherein the cell further comprises a modified carbohydrate synthesis pathway, glutamine synthase (GS) and/or dihydrofolate reductase (DHFR) as compared to a control cell of the same species.

The article of manufacture or kit may be accompanied by instructions for use.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### TSC1 Deletion Promotes Differentiation of B Cells into Plasma Cells and Increases Secretion of Antibodies Materials and Methods

Mice—

Wild type C57BL6 and BALB/C mice were purchased from Harlan Israel. CD19-Cre/XBP1^{f/f} (XBP1 KO), CD19-Cre/TSC1^{f/f} (TSC1 KO) and RERT/TSC1^{f/f} mice were obtained as described (15 and PMID 19454701). ROSA26-floxed stop-lacZ YFP reporter mice were purchased from Jackson laboratories (B6.129X1-Gt(ROSA)26Sor^{tm1(EYFP)Cos}/J). Other mice were generated by intercrossings. All CD19-Cre strains were crossed for 5 generations onto the YFP reporter strain and all RERT strains were crossed for 5 generations onto Balb/C background.

Antibodies and Reagents—

Rat anti-CD138-APC, rat anti-mouse CD45R/B220-PerCP, rat anti-mouse Ly6C-PE, rat anti-mouse CD5-PE and rat IgG2a-PerCP isotype control were purchased from BioLegend, Inc. (San Diego, Calif.). Rat anti-mouse Kappa-PE was purchased from Southern Biotechnology Associates, Inc (Birmingham, Ala.). Rat IgG2b isotype control was purchased from Ebioscience (San Diego, Calif.). Rapamycin was purchased from LC laboratories (Worburn, Mass.). LC3 conjugated HRP was purchased from Novus biological (Littleton, Colo.). Anti-TSC1 and anti-p62 antibodies were purchased from cell signaling (Danvers, Mass.). Anti-Ly6C blocking antibody (clone 1G7G10, Eur J Immunol. 2011 41(3):634-44). LPS (Sigma, cat no: L3755) was used at 20 μg/ml, carboxyfluorescein diacetate succinimidyl ester (CFSE) (Invitrogen) labeling (1 μM) was performed for 5 minutes at room temperature. Tamoxifen (Sigma, cat no: T5648) was dissolved in corn oil to 20 mg/ml. Knock out was induced by three consecutive s.c. injections of tamoxifen at 5 μl/g of mouse weight.

Cells Purification and Culture Conditions—

Mice were sacrificed and spleens, peripheral lymph nodes (pLN: axilar, inguinal and popliteal lymph nodes), mesenteric lymph nodes (MLN) and/or femur and tibiae were harvested in cold phosphate-buffered saline (PBS).

Splenocytes and LN cells were purified using mechanical shearing of the corresponding tissue with a syringe plunger followed by filtering through a 70 μm mesh cell strainer. Cells were centrifuged and red blood cells were lysed by ACK buffer Ammonium Chloride 8290.0 g/L; Potassium Bicarbonate 1000.0 g/L; EDTA 37.0 g/L) for 10 minutes at room temperature. Following two washes with PBS the splenocytes and LN cells were either analyzed directly by flow cytometry or used for mature B cell isolation.

Mature B cells were purified from the harvested spleens by anti-CD43 magnetic depletion (Miltenyi Biotec, Bergisch Gladbach, Germany) according to manufacturer's instructions. Following purification cells were plated at 1.5×10⁶ cells/ml in complete medium: RPMI 1640 (Invitrogen-Gibco) supplemented with 10% FBS (Biological Industries, Kibbutz Beit Haemek, Israel), 2 mM glutamine, 50 U/ml penicillin, 50 μg/ml streptomycin, 50 μM β-Mercaptoethanol, 25 mM 1× nonessential amino acids, and 1 mM sodium pyruvate (Biological Industries, Kibbutz Beit Haemek, Israel) and E. Coli LPS 20 μg/ml (Sigma L3755). Single cell suspension of pLN was prepared by forcing the tissue through a mesh cell strainer using a syringe plunger and subjected to staining and flow cytometry analysis. The harvested MLNs were rinsed in cold PBS and incubated with shaking for 45 minutes at 37° C. in a digestion solution containing 1 mg/ml Collagenase (Sigma), 0.33 mg/ml DNAse I (Roche diagnostics, Mannheim, Germany) and 0.1 mg/ml Dispase II (Sigma). Following incubation the tissue was gently scratched using a syringe plunger, resuspended, filtered through a 70 μm cell strainer and plated at 2×10⁶ cells/ml in complete medium. Stimulation was performed with 0.4 mg/ml APRIL (Peprotech/Tebu, Frankfurt, Germany) for 6 days. Rapamycin was dissolved in DMSO at 10 mg/ml and diluted in PBS to 100 μg/ml. Final concentration was 50 or 100 ng/ml.

Bone marrows (BM) cells were extracted from the harvested femur and tibiae purified from the surrounding muscle tissue. The intact bones were left for disinfection in 70% ethanol for 1 minute, washed twice in PBS, and transferred into a fresh dish containing RPMI 1640. Thereafter, both ends of each bone were cut with scissors and the marrow was flushed with 2 ml of RPMI 1640 using a syringe and 25-gauge needle. Clusters within the marrow suspension were disintegrated by vigorous pipetting, put on ice for 3 minutes to remove debris and treated with 5 ml of ACK red blood cells lysis buffer for 10 minutes at room temperature. Cells were than washed in PBS, counted, stained with the respected antibodies and analyzed by flow cytometry.

Immunoglobulin ELISA—

Immunoglobulin (Ig) concentrations in the sera of mice, in the culture supernatants of B cells purified from spleens or in the supernatant of MLNs were assessed by ELISA using SBA clonotyping system/HRP kits (Southern Biotechnology Associates, Inc., Birmingham, Ala.) according to manufacturer's instructions. Series dilutions were performed and the O.D. measurements at the linear phase of dilutions are presented.

Flow Cytometry Analysis—

Single cells were stained with mouse-conjugated antibodies or mouse isotype control for 30-45 minutes on ice. Propidium iodide (PI) labeling was performed by incubating the cells for 10 minutes with 50 mg/ml PI. Following staining, all cells were washed once with PBS containing 5% FBS (FACS buffer) and analyzed by BD LSRII flow cytometer (Becton Dickinson, Franklin Lakes, N.J.). Data was analyzed using FCS Express V3 analysis software (De Novo, Calif., USA).

Cells that were labeled with CFSE were analyzed following 3 days of stimulation using the same procedure.

Cell Sorting—

Cells from the different ROSA26-floxed stop-lacZ YFP reporter mice were stained with APC-conjugated anti-CD138 antibody for 30-45 minutes on ice. Following incubation cells were washed once with FACS buffer and sorted for using the BD FACSAria™ II.

Preparation of Cells for Transmission Electron Microscopy—

Cells were sorted from the bone marrow based on YFP and CD138 expression. Cells were collected, fixed in 2.5% Glutaraldeyde, 2% paraformaldehyde in 0.1 M Cacodylate buffer (pH 7.4) for 2 hours at room temperature, rinsed 4 times, 10 minutes each, with cacodylate buffer and post fixed and stained with 1% osmium tetroxide, 1.5% potassium ferricyanide in 0.1 M cacodylate buffer for 1 hour. All chemicals were purchased from Fluka or Sigma-Aldrich at analytical grade. Cells were then washed 4 times with cacodylate buffer followed by dehydration in increasing concentrations of ethanol consisting of 30%, 50%, 70%, 80%, 90% and 95%, for 10 minutes each step followed by 3 times 100% anhydrous ethanol, 20 minutes each and twice with propylene oxide, 10 minutes each. Following dehydration, the cells were infiltrated with increasing concentrations of Agar 100 resin in propylene oxide, consisting of 25, 50, 75, and 100% resin for 16 hours each step. The cells then were embedded in fresh resin and let to polymerize in an oven at 60° C. for 48 hours. The cells embedded in the blocks were sectioned with a diamond knife on an LKB 3 microtome and ultrathin sections (80 nm) were collected onto 200 Mesh, thin bar copper grids. The sections on the grids were sequentially stained with Uranyl acetate and Lead citrate for 10 minutes each and viewed with Tecnai 12 TEM 100 kV (Phillips, Eindhoven, the Netherlands) equipped with MegaView II CCD camera and Analysis® version 3.0 software (SoftImaging System GmbH, Miinstar, Germany).

IgA Immonospot Analysis—

IgA-secreting plasma cells in MLN cells were determined by ELISpot^{plus} for mouse IgA (MABTECH, Sophia Antipolis, France) according to manufacturers' instructions. Briefly, the ELISpot plate was pre-wetted by adding 50 ml 70% ethanol per well for 2 minutes, and coated overnight at 4° C. with total anti-IgA diluted in PBS. Following 5 washes with sterile PBS, the plate was blocked with complete medium (RPMI 1640 containing 2 mM glutamine, 50 U/ml penicillin, 50 μg/ml streptomycin, 50 μM β-Mercpatoethanol, 25 mM 1× nonessential amino acids, and 1 mM sodium pyruvate) supplemented with 10% FBS for 30 minutes at room temperature. Cell suspensions were added to the ELISpot (50,000 cells/200 ml/well) plate and the plate was incubated in a 37° C. humidified incubator with 5% CO₂ for 16-24 hours. Following incubation, the plate was washed 5 times with sterile PBS and biotinylated with anti-IgA to 1 mg/ml in PBS containing 0.5% FBS for 2 hours at room temperature. After washing in sterile PBS, streptavidin-ALP (1:1000) in PBS-0.5% FBS was added to the plate. Following incubation of 1 hour at room temperature the plate was washed with sterile PBS and the individual IgA secreting cells were visualized by addition of BCIP/NBT-plus substrate. The reaction was stopped by extensive washing in tap water.

Metabolic Labelling, Pulse-Chase Analysis and Immunoprecipitation—

Pulse labelling was performed as previously described (6). Briefly, following 45 minutes of starvation in methionine/cysteine-free Dulbecco's modified Eagle's medium (Biological Industries Beit Haemek, Israel) the cells were metabolically labelled for 20 minutes with [³⁵S] methionine/cysteine (7.5 μCi/500 μL) (Perkin Elmer, USA) at 37° C. To compare the incorporation of radiolabeled ³⁵S-methionine, lysates of an equal number of metabolically labelled cells were prepared in SDS 1% diluted in lysis buffer (Tris pH 8, 50 mM, NaCl 200 mM, MgCl₂ 20 mM and 1% NP-40, 3 μL/mL normal rabbit serum, 10 μL/mL BSA 0.1% and protease inhibitors). Goat anti-mouse isotype specific antibodies were used for immunoprecipitation. Bands were quantified by phosphor-imager.

Quantitative Real-Time PCR (qRT-PCR)—

Total RNA was isolated using TriReagent (Sigma). RNA samples were treated with DNAseI and purified by ethanol precipitation. 1 μg of total RNA was reversed transcribed into cDNA using a Reverse-iT first strand synthesis kit with random decamers (Fermentas). Real time PCR reactions were performed using a SYBR Green PCR Master Mix (Finnzyme) and CFX connect real-time system (Bio-Rad). Ly6C1 expression level was determined in all samples as compared to Ubiquitin C (UBC) controlling for any variability in RNA input. The following primers were used for qPCR:

Ly6C-Forward: GCA GTG CTA CGA GTG CTA TGG (SEQ ID NO: 29); Ly6C-Reverse: ACT GAC GGG TCT TTA GTT TCC TT (SEQ ID NO: 30); UBC-Forward: CAG CCG TAT ATC TTC CCA GAC T (SEQ ID NO: 31); and UBC-Reverse: CTC AGA GGG ATG CCA GTA ATC TA (SEQ ID NO: 32). Thermal cycling conditions included initial denaturation at 95° C. for 3 minutes followed by 39 cycles of 3 seconds at 95° C., 30 seconds at 60° C., followed by 10 seconds at 95° C., 5 seconds at 65° C. and 50 seconds at 95° C.

Analysis of XBP-1 Deletion by PCR—

Mature B cells from DKO/YFP mice were isolated from spleens as described above and sorted for YFP expression. DNA was purified using proteinase K digestion followed by precipitation with isopropanol and wash with 70% ethanol. PCR was performed with the mix of LAROVA, (Jena Germany) using BioRad mycycler. The following primers were used

INT1-S: CTTTGTGGTCGTAGGGTAGGAACC (SEQ ID NO: 33);

3′lox-S: ACTTGCACCAACACTTGCCATTTC (SEQ ID NO: 34); and

3′lox-A: CAAGGTGGTTCACTGCCTGTAATG (SEQ ID NO: 35).

Thermal cycling conditions included 40 cycles of annealing at 58° C., elongation at 72° C. for 30 sec and denaturation at 95° C. for 30 sec.

Western Blot Analysis—

Cells were washed twice with cold PBS and whole cell lysates were prepared in RIPA buffer (25 mM Tris.HCl pH 7.6, 150 mM NaCl, 1% NP-40, 1% sodium deoxycholate, 0.1% SDS, 1 mM Na3VO4, 50 mM NaF, 10 mM Sodium glycerophosphate, 10 mM Sodium Pyrophosphate and protease inhibitors (Sigma-Aldrich Cat No: S8820). The lysates were cleared by centrifugation. Total protein concentration was determined using the BCA Protein Assay Reagent Kit (Pierce). Following SDS-PAGE analysis under reducing conditions, gels were electro-transferred to nitrocellulose membranes. Thereafter, membranes were blocked in Tris-buffered saline containing 0.1% Tween® 20 (TBST), 5% milk powder and probed with the specific antibodies, followed by secondary horseradish peroxidase-conjugated antibodies. ECL reagent (Biological Industries, Beit Haemek, Israel) was used to develop the blots by chemiluminescence. p97 was used as normalization control.

Results:

TSC1 Deletion Promotes Differentiation of B Cells into Plasma Cells

In order to characterize the effect of deletion of TSC1 on differentiation of PCs, knock-outs of TSC1 (referred to herein as TSC1 KO), XBP-1 (referred to herein as XBP-1 KO) and TSC1 XBP-1 double knock-out mice (referred to herein as DKO) were used.

As demonstrated in FIG. 1, comparison of serum Ig titers in XBP-1 KO and DKO mice relative to wild type (wt) and TSC1 KO mice indicated that deletion of XBP-1 results in reduced levels of total Ig and specifically IgM and IgG1. However, in stark contrast to the XBP-1 KO mice, the DKO mice exhibited a marked elevation in serum Igs, particularly IgA. In fact, IgA titers of a few of the DKO mice tested were equivalent to the titers of wt and TSC1 KO mice (FIG. 1).

This data suggested that mTOR activation by deletion of TSC1 dominates the requirement for XBP-1 for antibody secretion, or that IgA-producing PCs can potentially escape XBP-1 deletion. In order to address the latter option, all the conditional KO strains were crossed to a ROSA26-STOP-YFP knocked-in reporter, which is expressed only upon Cre-mediated recombination (13). In total, four strains were generated; wt/YFP, XBP-1 KO/YFP, TSC1 KO/YFP and DKO/YFP. YFP positive and negative cells were sorted from MLN of DKO/YFP mice to ensure that the YFP expression represents a successful recombination of both TSC1 and XBP-1 genes. As shown in FIG. 2A, Western blot analysis indicated that TSC1 was not expressed in the YFP-positive fraction, substantiating the YFP as a reliable marker of TSC1 recombination. As further shown in FIG. 2B, PCR analysis for the XBP-1 locus from the same fractions demonstrated full recombination of the XBP-1 floxed gene. Similar results were obtained for B cells sorted from the various lymph nodes (data not shown). Taken together, the YFP expression reliably indicates CD19-driven recombination of both TSC1 and XBP-1 loci.

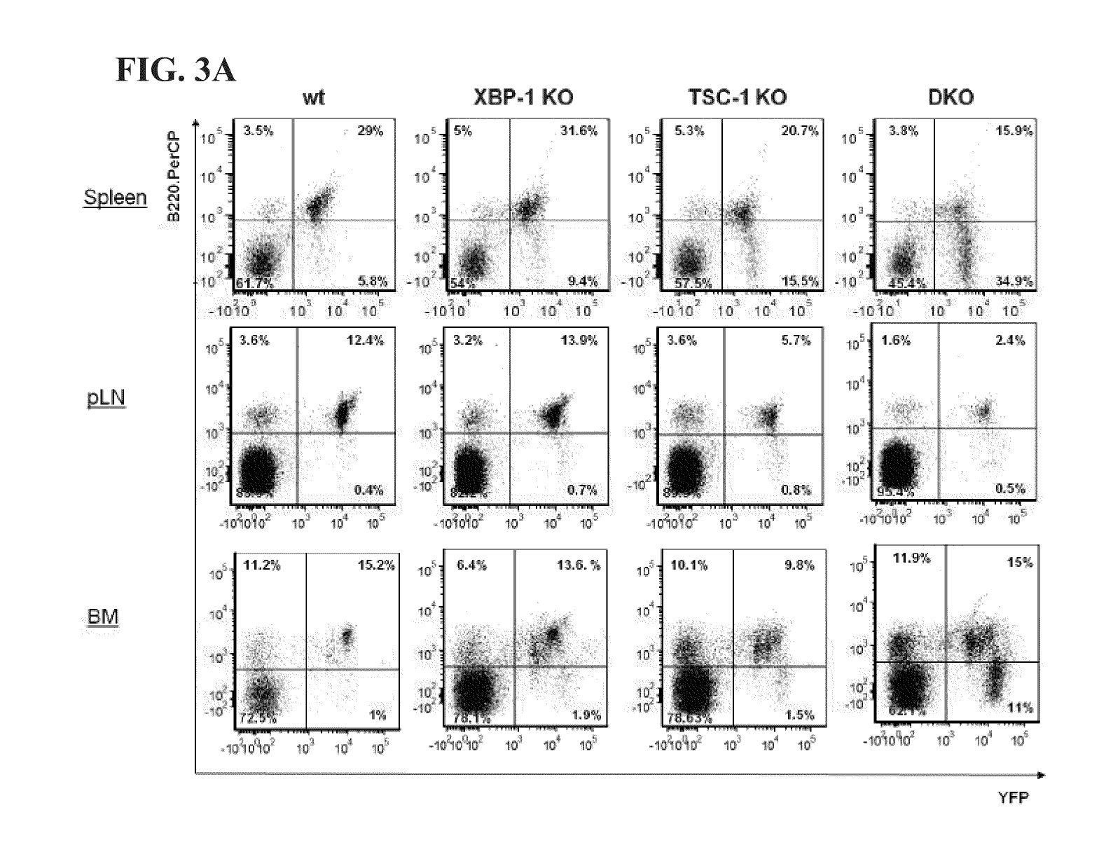

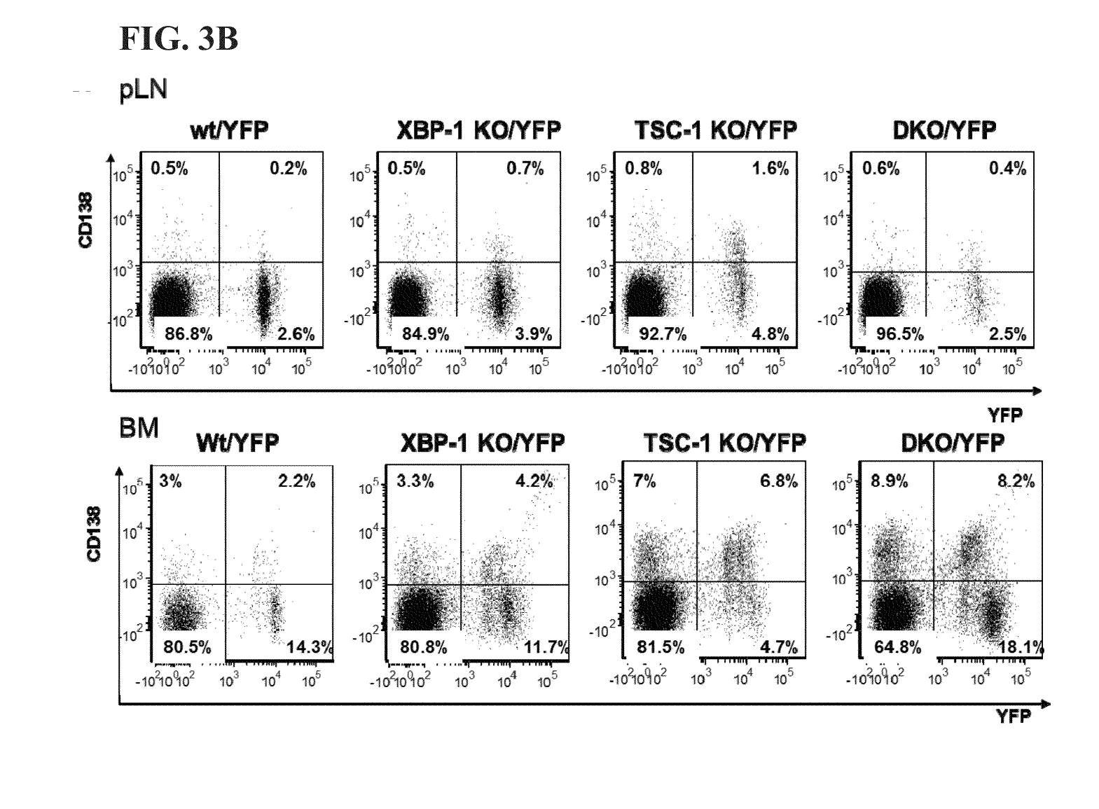

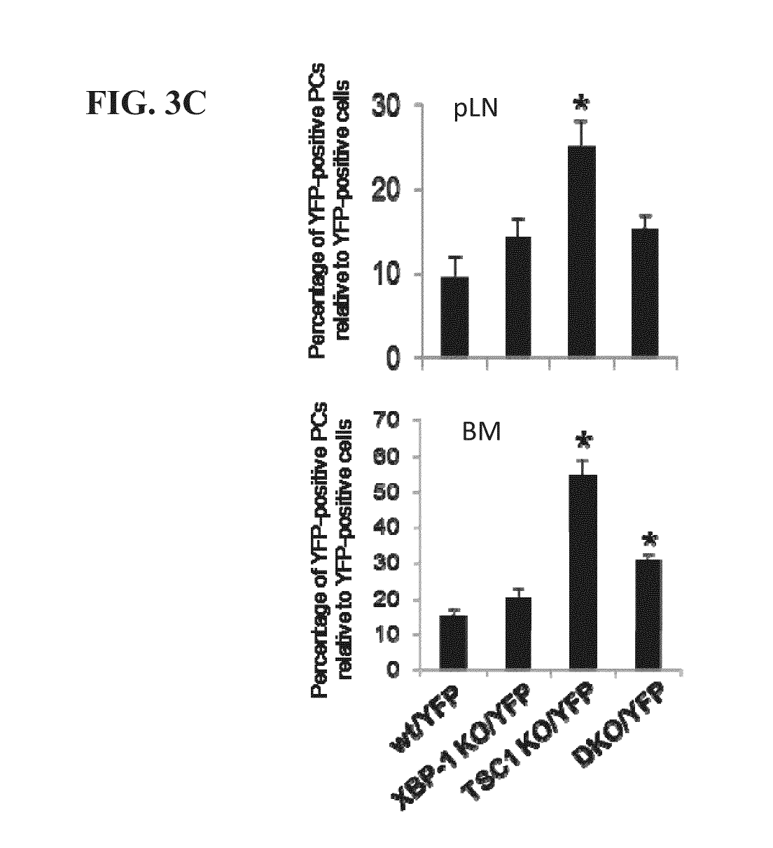

In the next step, single cell suspensions of spleen, peripheral lymph nodes (pLN) and bone marrow (BM) from all four strains were analyzed for YFP (an indicator for CD19 expression), B220 and the PC marker CD138. As demonstrated in FIG. 3A, three populations of B cells can be clearly distinguished from one another YFP⁺B220⁻ (PC cells), YFP⁻B220⁺ and YFP⁺B220⁺ (non-PC B cells). In accordance with previous studies indicating that lack of XBP-1 does not affect B cell maturation (5), wt and XBP-1 KO mice displayed similar B cell distribution in the naïve spleen and pLN cells. However, a higher representation of PC cells (YFP⁺B220⁻ cells) was observed in naïve spleen cells of TSC1 KO and DKO mice (FIG. 3A).

In pLN cells, TSC1 deletion reduced the number of YFP⁺B220⁺ cells, which is in line with the defects in B cell maturation. The co-deletion of XBP-1 and TSC1 conferred a further reduction in YFP⁺B220⁺ cell numbers. Unexpectedly, an enrichment of surface CD138 in the YFP pLN cells obtained from TSC1 KO and DKO was detected, although DKO mice contained fewer YFP⁺B cells (FIGS. 3B-C). This data indicates that while TSC1 is required for proper B cell maturation, its absence promotes the expansion of PCs in pLN.

It is known that long-lived PCs reside primarily in the BM (17). Because early B cell development to the immature state also occurs in the BM, the B220/YFP markers distinguish between PCs (B220⁻/YFP⁺) from non-PC B cells (B220⁺/YFP⁻ or B220⁺/YFP⁺). In the DKO mice a clear enrichment was seen for YFP⁺B220⁻ cells. Analysis of the expression of the PC marker CD138 (FIGS. 3B-C) showed that while in BM cells of wt and XBP-1 KO mice the majority of YFP⁺ cells were CD138 negative, a larger portion of the YFP⁺ cells were positive for CD138 in the TSC1 KO similar to pLN cells. A more modest enrichment in CD138⁺ population relative to wt and XBP-1 KO was observed in BM cells of DKO mice (FIG. 3C). Taken together, deletion of TSC1 confers enrichment in PCs in the pLN and BM. The milder phenotype of the DKO suggests that the mechanism is only partially mediated by XBP-1.

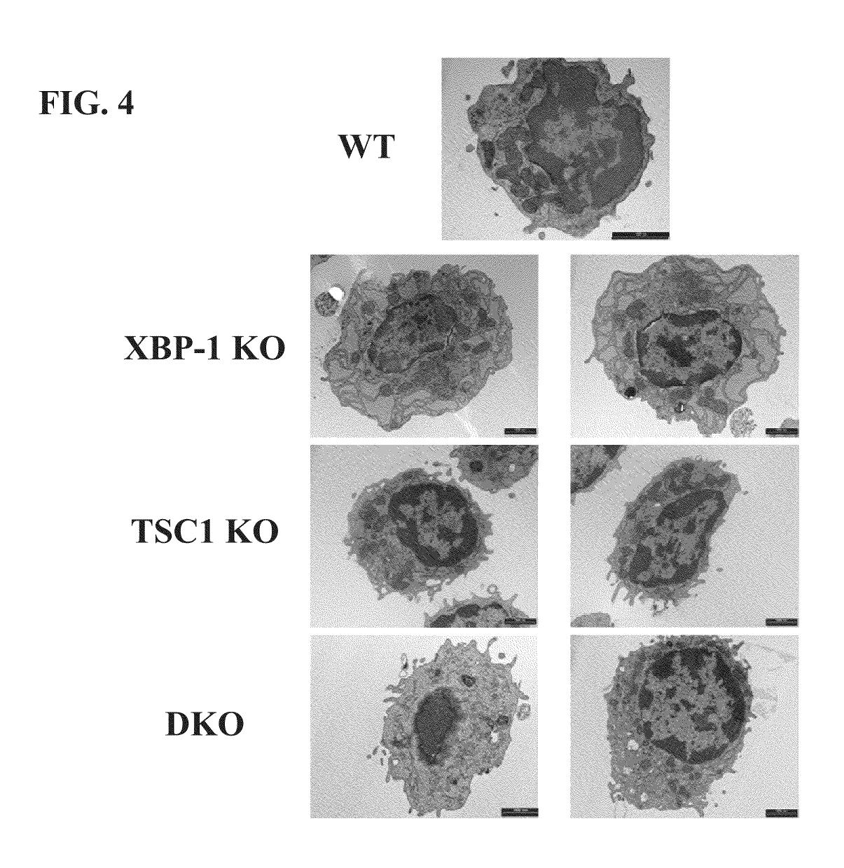

A recent study utilizing Blimp-1/GFP knock-in mice to enumerate and characterize the role of XBP-1 in PC development demonstrated normal development of PCs in the absence of XBP-1, however, these cells exhibited a defect in ER morphology (5). In order to test the effect of TSC1 on ER morphology, PCs from the BM were sorted based on the B220/YFP markers and processed for transmission electron microscopy. In accordance with the previous results, XBP-1-deficient PCs exerted a dilated ER morphology. Remarkably, as demonstrated in FIG. 4, the vast majority of the DKO PCs had an indistinguishable ER morphology from wt or TSC1 KO PCs. This suggests that mTOR either promotes corrective measures for ER homeostasis under conditions of impaired UPR, or it selects for PCs with a functional ER.

TSC1 Deletion Promotes IgA Secretion in the Absence of XBP-1.

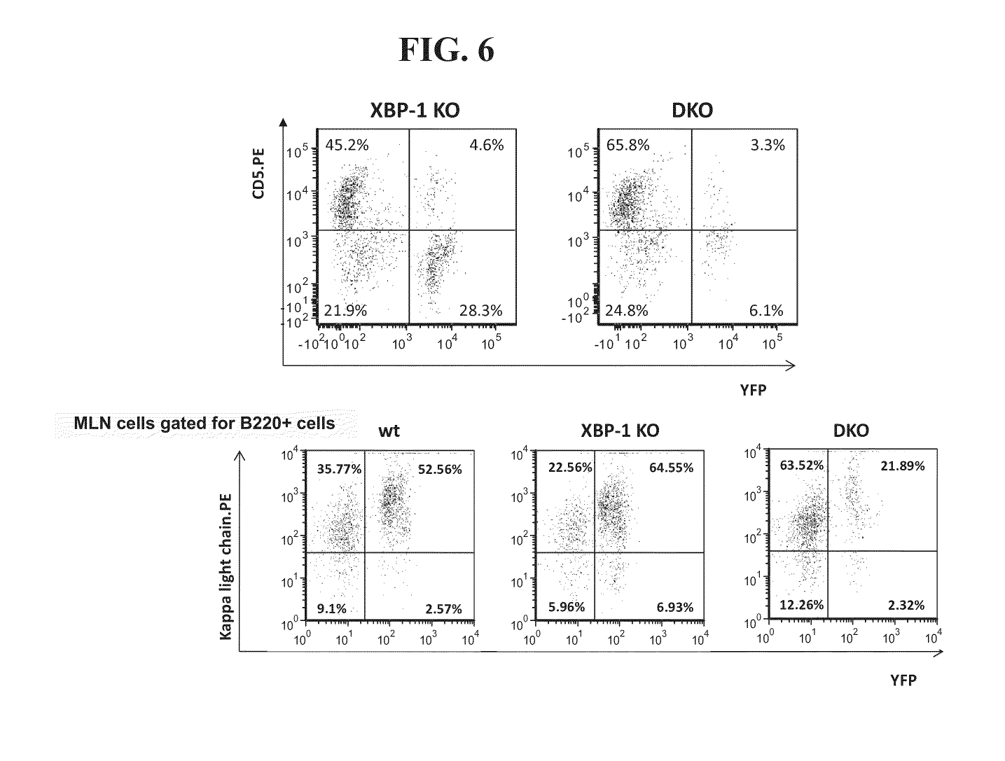

In order to specifically assess the contribution of mTOR activation to IgA synthesis and secretion, MLN cells isolated from the various YFP strains were stimulated by APRIL, a TNF superfamily member cytokine. At the time of extraction, most of the MLN B cells isolated from wt mice were YFP⁺. The YFP⁻ population mostly comprised of CD5⁺CD19⁻ cells (data not shown). Following stimulation of cells isolated from wt mice, intracellular light chain content was higher in the YFP⁺ as compared to the YFP⁻ cells and CD138 was expressed exclusively in the YFP⁺ compartment, indicating that antibody forming cells (AFC) are mostly present in the YFP⁺ population (FIGS. 5-6). In comparison to cells isolated from wt and XBP-1 KO mice, stimulation of cells isolated from DKO mice resulted in significant reduction in the proportion of YFP⁺ cells relative to the YFP⁻ cells. However, consistent with the findings in the BM and pLN B cells as detailed hereinabove, the percentage of the CD138⁺YFP⁺ cells relative to the entire YFP⁺ population was elevated in cells isolated from DKO mice relative to cells isolated from XBP-1 KO mice (FIG. 5).

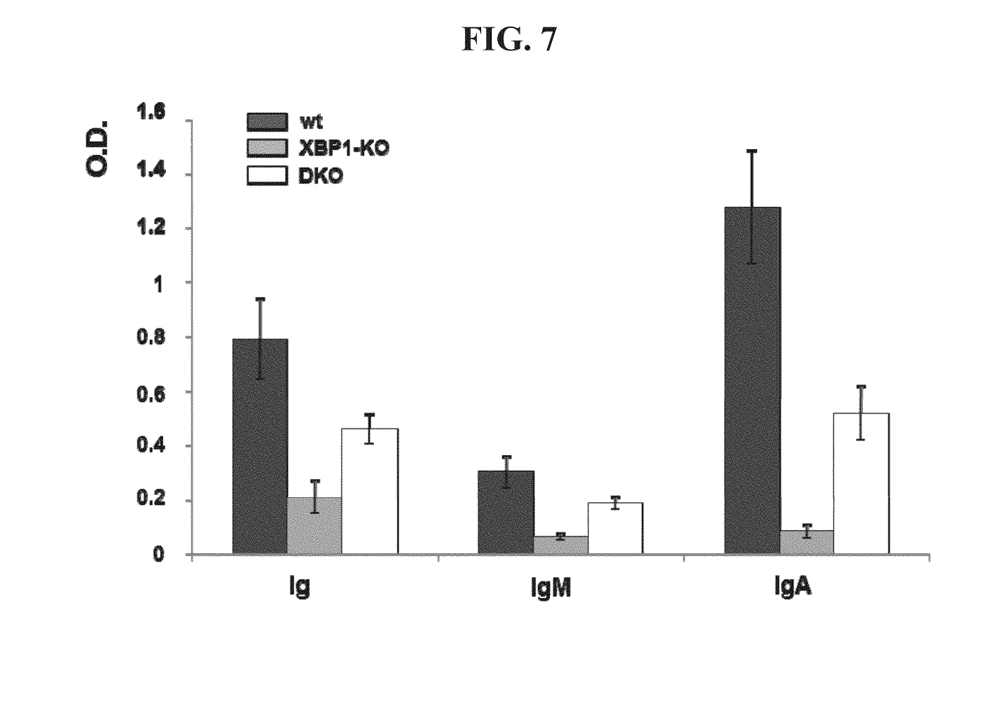

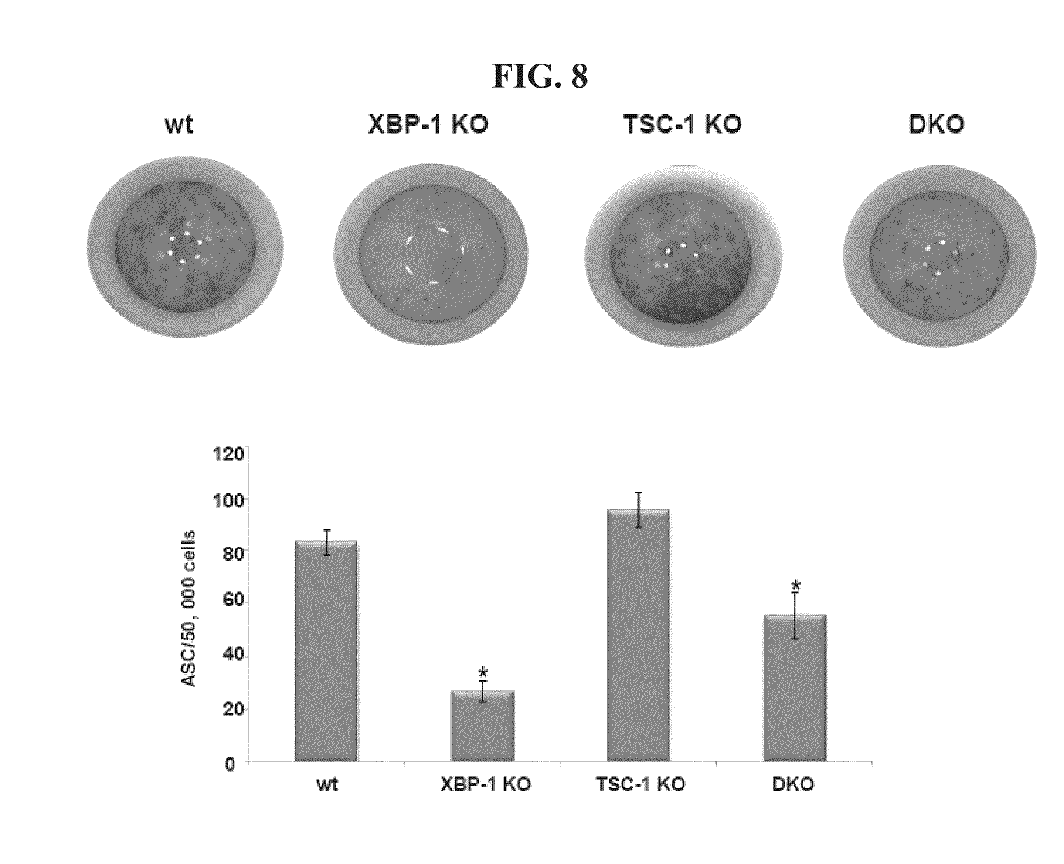

Despite the reduction in total YFP⁺ B cell numbers following APRIL stimulation observed in the DKO cell cultures, analysis of the IgA content in the culture supernatants following stimulation showed significantly higher IgA levels in the DKO cell cultures as compared to the XBP-1 KO cell cultures (FIG. 7). This observation was further supported by an IgA ELISPOT assay performed on MLN YFP⁺ cells sorted following stimulation which showed significantly higher numbers of DKO-derived AFC as compared to XBP-1 KO (FIG. 8).

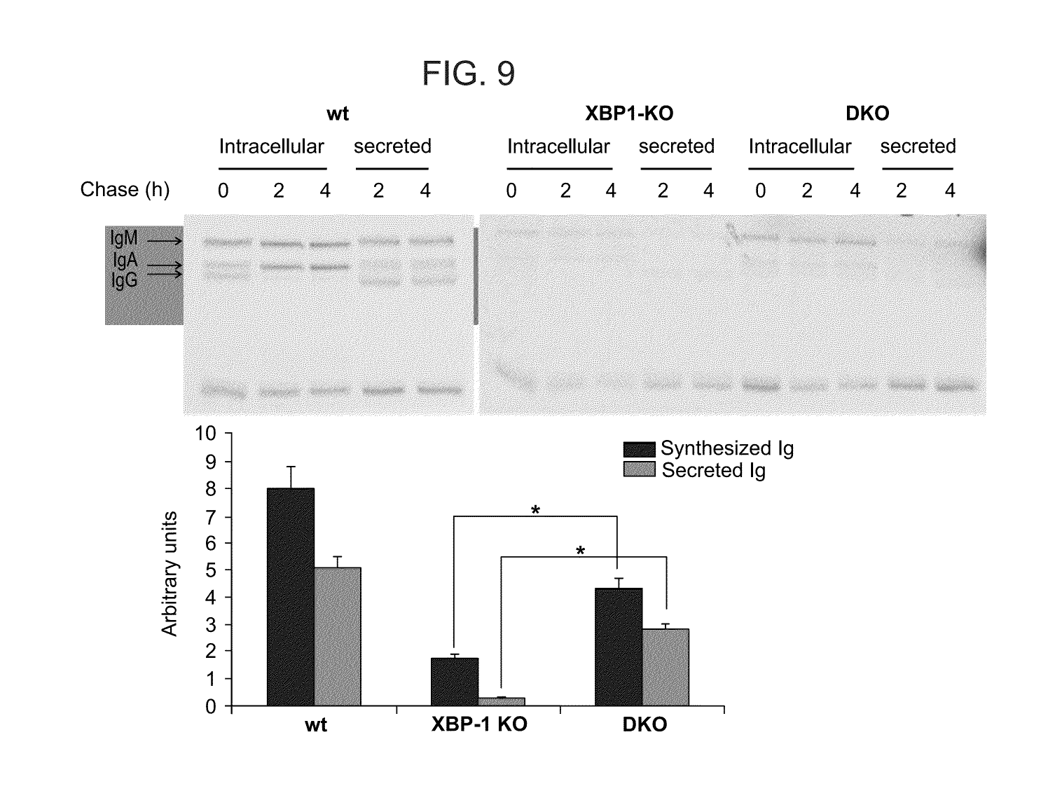

mTOR promotes protein synthesis through various mechanisms, such as phosphorylation of 4E-BP and S6K1, thus the capacity of DKO cells to synthesize Ig molecules was compared to the XBP-1 KO cells on a per cell basis. To this end, equal numbers of MLN YFP⁺ cells from wt, XBP-1 KO and DKO mice sorted following APRIL stimulation were subjected to pulse-labeling with ³⁵S-methionine followed by a chase period. As demonstrated in FIG. 9, an increased synthesis of IgA heavy chains was observed in DKO cells as compared to XBP-1 KO cells, as well as higher levels of radioactive IgA in the culture supernatants. Furthermore, the addition of Rapamycin significantly reduced the levels of IgA secreted to the culture supernatants by the DKO cells, indicating that pharmacological inhibition of the mTOR in DKO cells retrieves the XBP-1 KO phenotype (FIG. 10). Taken together, in the absence of XBP-1, mTOR activation leads to a higher secretion of IgA, and promotes differentiation into AFCs in a cell autonomous manner.

In order to elucidate whether the observed increase in IgA secretion resulting from mTOR activation can be attributed to the abnormal B cell development and/or to a direct effect on specific elements in the IgA secretion process, the present inventor has tested whether knockout of TSC1 following B cell maturation also affects Ig synthesis and differentiation into PCs. To this end, a RERT mouse strain was used which expresses an IRES-CRE-ER element knocked into the heavy subunit of RNA polymerase II thus allowing activation of Cre by tamoxifen (19). Floxed XBP-1, TSC1 and TSC1/XBP-1 were crossed to the RERT strain (termed RERT/XBP-1 KO, RERT/TSC1 KO and RERT/DKO, respectively). Tamoxifen-treated XBP-1 KO mice were used as controls. Eight days following tamoxifen treatment, a period of time in which efficient deletion of the floxed genes was observed and no aberrations were seen for B cell development, splenic B cells were isolated and subjected to stimulation with LPS.

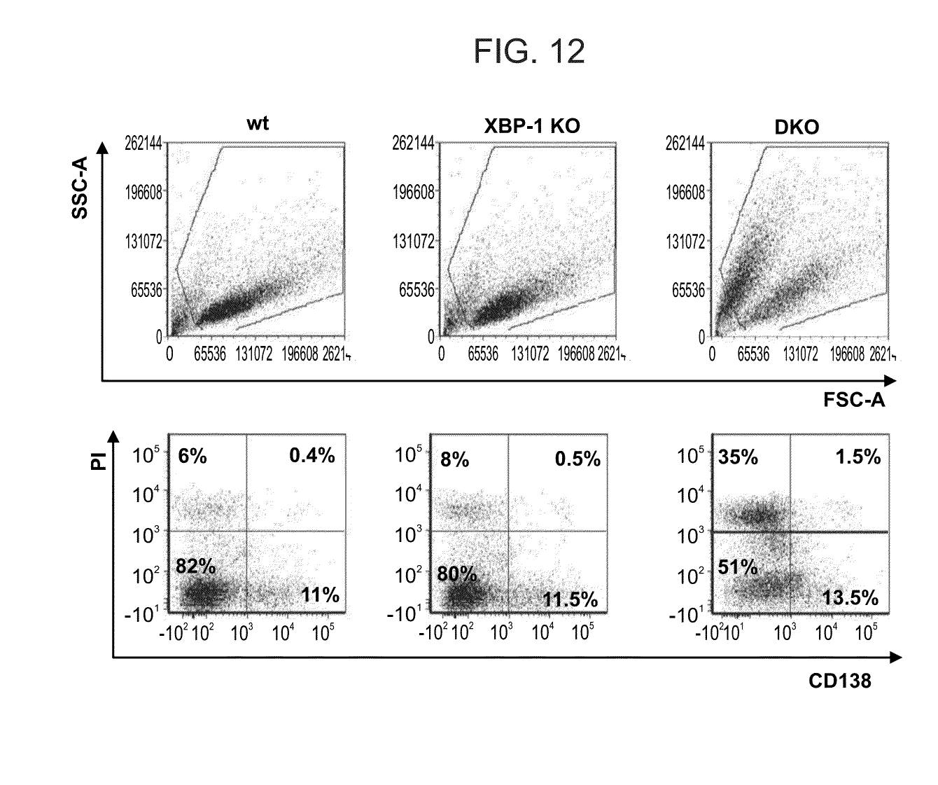

CFSE dilution analysis indicated that deletion of TSC1 has no effect on proliferation of the splenic B cells (FIG. 11). However, cell viability of splenic B cells isolated from RERT/DKO mice was severely compromised, as evident from their forward and side scatter properties and the incorporation of PI (FIG. 12). Analysis of the PI⁻ live cells, demonstrated that TSC1 deletion results in enrichment in the number of CD138⁺ cells (FIG. 12).

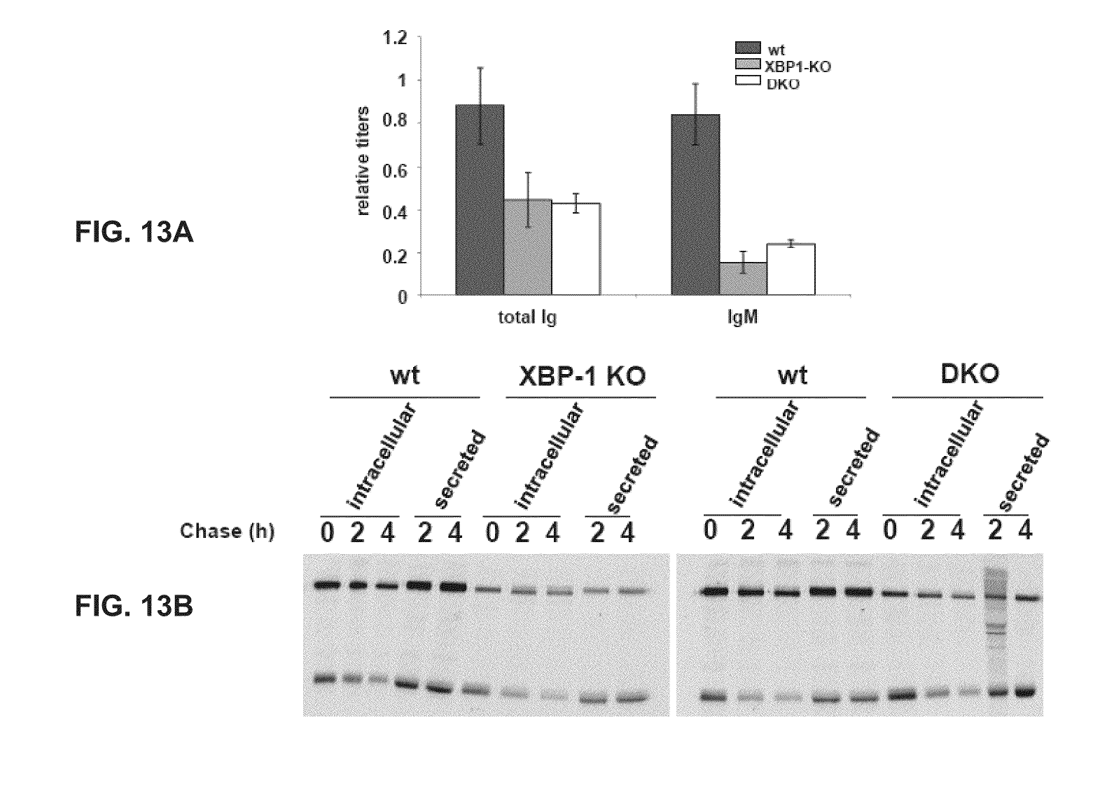

To examine whether TSC1 deletion also promotes Ig secretion, both Ig ELISA and pulse chase analysis were employed (FIGS. 13A-B). Ig ELISA revealed that the levels of IgM in the supernatants of LPS-stimulated splenic B cells were significantly higher in cultures of RERT/DKO cells as compared to RERT/XBP-1 KO cells (FIG. 13A). This result is highlighted given the almost two-fold reduced survival of RERT/DKO cells. Pulse-chase analysis for an equal number of live cells demonstrated increased production of IgM heavy chains and increased secretion of IgM from LPS-stimulated splenic B cell cultures of RERT/DKO cells as compared to RERT/XBP-1 KO cells (FIG. 13B).

Taken together, these data demonstrate that activation of mTOR increases PC differentiation and induces synthesis and secretion of immunoglobulins.

Ly6C is Involved in Promoting IgA Secretion in an XBP-1 Independent Mechanism.

The effect of mTOR activation by TSC1 deletion on PC development and function can be attributed to both direct and indirect pathways. Thus, mTOR activation may directly enhance Ig synthesis and expression (as shown hereinabove), inhibit degradative pathways, such as autophagy (20), and/or indirectly promote expression of molecules signaling for enhanced PC differentiation.

The direct effect of mTOR on protein synthesis and trafficking cannot fully explain the increased expression of CD138 in the TSC1 KO (presented hereinabove), suggesting that mTOR integrates signals into the physiological PC program.

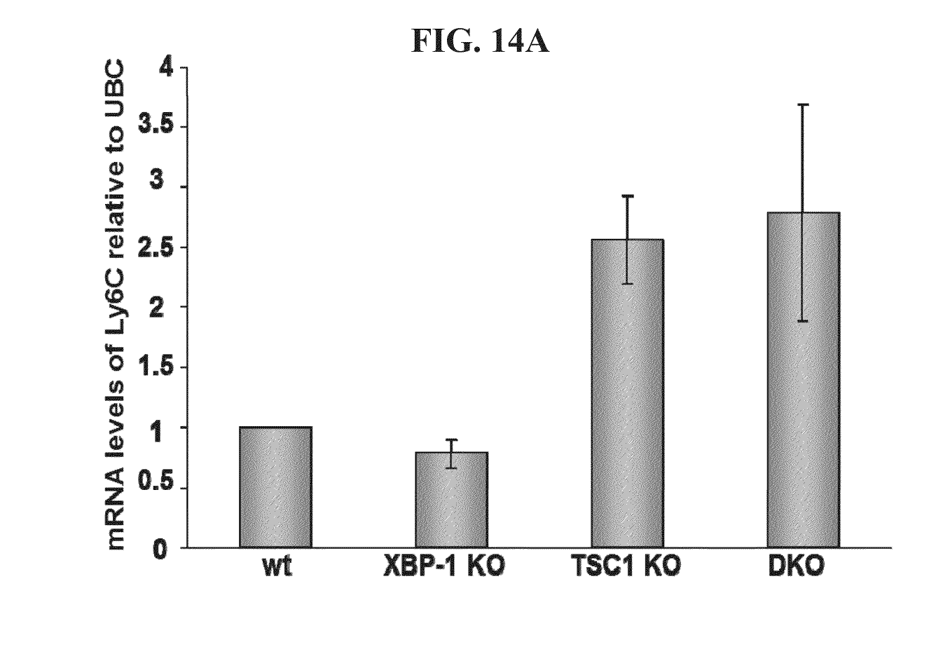

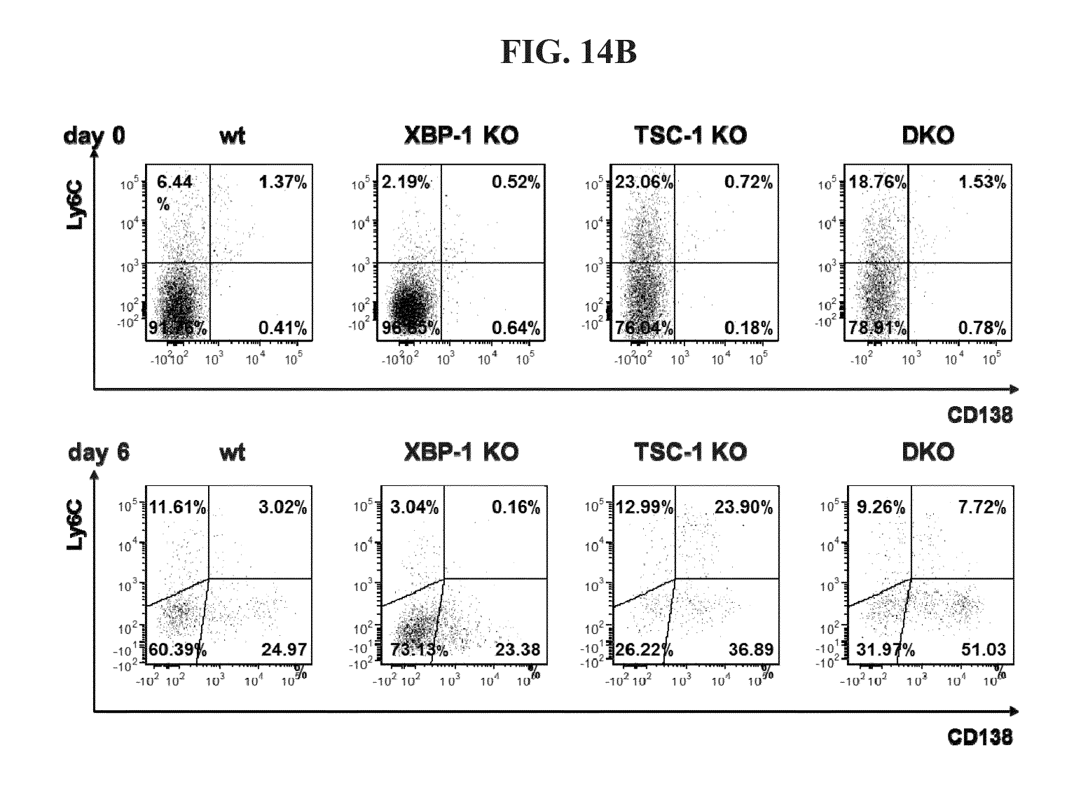

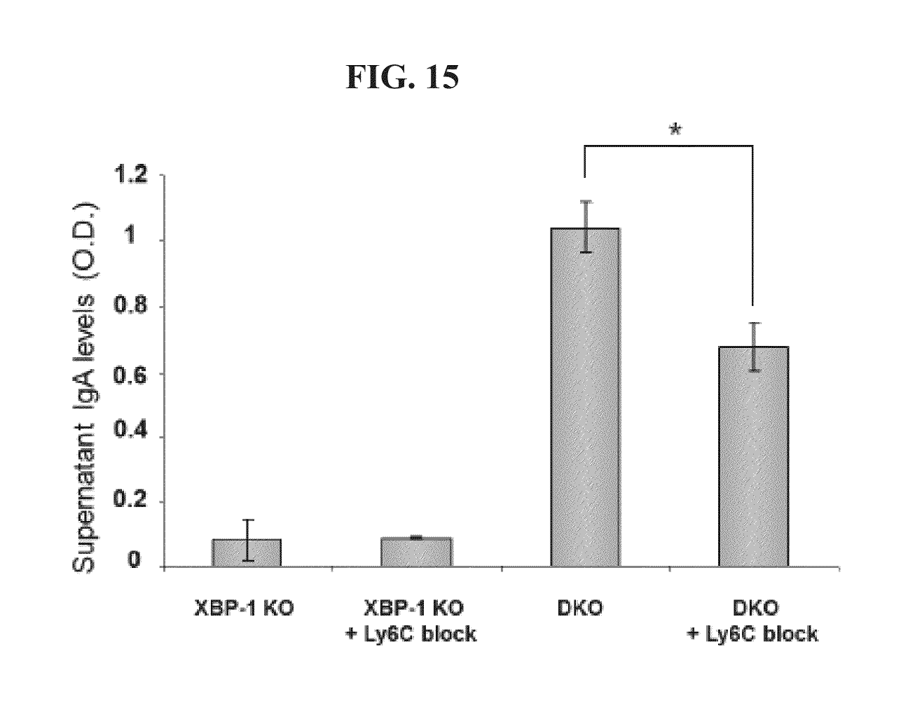

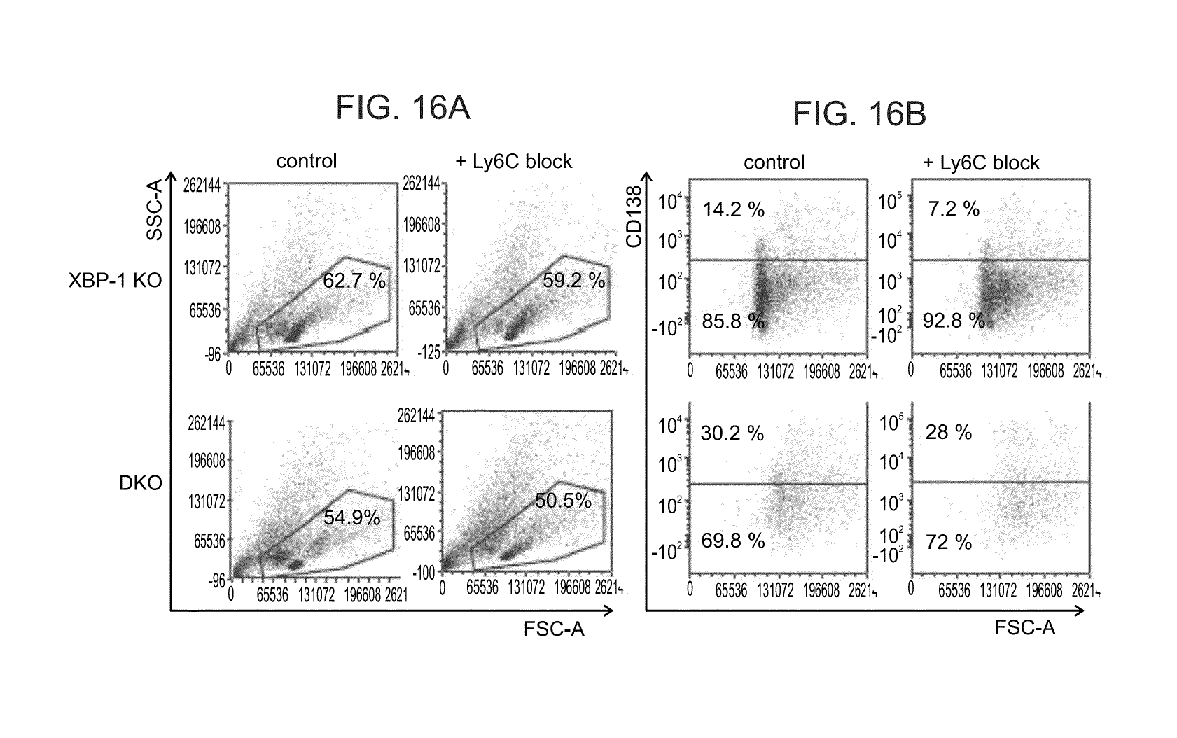

Ly6C is a cell surface glycoprotein expressed mainly in hematopoietic cells. Ly6C differentiates between PCs and other B cell subsets, wherein the highest expression documented is in IgA secreting PCs (21). To elucidate the role of Ly6C in PC differentiation in the absence of TSC1, the expression of Ly6C in APRIL-stimulated YFP⁺ cells isolated from MLN of the different mouse strains was evaluated both in the RNA and the protein levels. Very few YFP⁺ DKO and TSC1 KO MLN cells survive the APRIL stimulation relative to wt and XBP-1 KO cells. Regardless, as demonstrated in FIGS. 14A-B quantitative-PCR analysis revealed a 2.5 fold higher Ly6C mRNA levels in DKO cells as compared to XBP-1 cells and flow cytometry analysis revealed a strong increase in the percent of Ly6C⁺ cells on the YFP-gated cells in TSC1 KO and DKO cells relative to wt and XBP-1 KO cells. Furthermore, addition of a blocking antibody to Ly6C reduced IgA secretion by 35% in DKO cells asserting Ly6C role in IgA secretion by these cells (FIG. 15). The antibody did not affect the amount of YFP⁺ cells nor their viability (FIGS. 16A-B). These results indicate that mTOR activation alters the PC program.

Taken together, these data indicate that activation of mTOR by TSC1 deletion compromises B cell viability when stimulated to become AFCs. However, cells that endure the process possess a better secretory capacity due to increased Ig synthesis and expression of specific elements in the PC program, such as Ly6C. Strikingly, these activities do not require the IRE1/XBP-1 arm of the UPR.

### DISCUSSION

To explore the relationship between mTOR and UPR in PC development and function, mice conditionally deleted for XBP-1 and/or TSC1 in their B cell lineage were generated. Deletion of TSC1 enhanced Ig synthesis and promoted differentiation into PCs in an UPR-independent mechanism, as evident by increased percentage of PCs and higher Ig titers in TSC1/XBP-1 double knockouts (DKO) relative to XBP-1 KO. Abnormal endoplasmic reticulum morphology, typically seen in XBP-1 KO PCs, was milder in DKO counterparts.

Ligation of Ly6C, a cell surface glycoprotein, promotes Ig secretion. Ly6C expression was specifically enriched in stimulated TSC1 KO and DKO B cells in a manner that further contributed to the enhanced Ig secretion from DKO cells. This reveals a functional overlap between mTOR and UPR in promoting Ig secretion from PCs. The mechanism entails control of protein synthesis and expression of accessory molecules, such as Ly6C.

To conclude, deletion of TSC1 promotes the differentiation of B cells into PCs and improves their secretory capacity. The mechanism does not require XBP-1, as DKO mice display IgA titers comparable to wt and significantly higher than those of XBP-1 KO. B cells of DKO mice generated higher levels of Ig molecules than XBP-1 KO B cells upon stimulation. Remarkably, mTOR activation corrected the distended ER morphology of XBP-1 KO PCs. Finally, Ly6C was identified as a downstream target of mTOR activation, which contributes to the mTOR bypassing of the UPR for antibody secretion.

### Example 2

### The Effect of TSC2 Deletion on Recombinant Protein Production

Materials and Methods:

Cell Lines and Culture Conditions—

293T cells (ATCC no. CRL-3216 denoted herein as HEK293) cells were cultured in DMEM supplemented with 10% FBS, pen-strep antibiotics and sodium pyruvate. CHO-K1 cells (ATCC no. CCL-61, denoted herein as CHO) were cultured in DMEM/F12 medium supplemented with 10% FBS, pen-strep antibiotics and sodium pyruvate.

Pulse-Chase Analysis—

Pulse-chase analysis using ³⁵S-methionine labeling. Cells were labeled for 30 min and chased up to 2.5h. GFP secretion was evaluated by immunoprecipitation with anti-GFP polyclonal antibody from the cell extract and from the supernatants (intracellular and secreted, respectively).

Generation of HEK293-GFP-Fc and CHO-GFP-Fc Cells—

GFP-Fc was generated by cloning EGFP after the signal peptide of H-2Kb followed by the human Fc portion of IgG1. The vector also contains puromycin resistance gene, a modification of pFUSE-hIgG1-Fc1 (Invivogen). HEK293 and CHO cells were transfected with the GFP-Fc vector using Transit 2020 (Mirus Bio, Madison, Wis.) according to manufacturer's instructions. Two days following transfection cells were treated with 2 μg/ml of puromycin for three days. Cells were then subjected to sorting based on GFP fluorescence expanded in culture.

Generation of TSC2 KO in HEK293-GFP-Fc and CHO-K1-GFP-Fc Cells—

In order to generate the knockout of TSC2 in HEK293T and CHO cells that stably express GFP-Fc, a CRISPR vector directed against the TSC2 gene was generated. The vector used was pX330 which expresses a FLAG-tagged Cas9 protein driven from a CMV promoter and the gRNA driven from the U6 promoter (Addgene, Cat. No. 42230. gRNA sequences were cloned into pX330 vector using the Zhang lab protocol (Broad Institute, Cambridge, Mass.). Briefly, pX330 was digested with BbsI and dephosphorylated by SAP. 5′ phosphorylated gRNA duplex containing the 20 bp targeting sequence was used for ligation into the digested pX330 vector. Clones were digested with NdeI which excises the insert. Successful insertion of the gRNA sequence into the pX330 backbone results in a bigger fragment that is resolved by poly acril amide gel (PAGE). Positive clones were sent for sequencing using the U6 primer 5′-gactatcatatgcttaccgt-3′ (SEQ ID NO: 36). The gRNA sequence used for targeting TSC2 in HEK293 was 5′-AACAATCGCATCCGGATGAT-3′ (SEQ ID NO: 28) which is directed against exon 3. The gRNA sequence used for targeting TSC2 in CHO cells was 5′-tcttcgtagggatggcactc-3′ (SEQ ID NO: 25), which targets exon 10.

HEK293-GFP-Fc and CHO-K1-GFP-Fc cells were transfected with the pX330 containing the respective gRNA using Transit 2020 (Mirus Bio, Madison, Wis.) according to manufacturer's instructions. The cells underwent two consecutive transfections. A week following the second transfection into the cells were analyzed by flow cytometry for GFP fluorescence and assessed biochemically for TSC2 expression.

Biochemical Assessment of TSC2 Expression—

A week following the second transfection with pX330 containing the respective gRNA cells were harvested and lysed in RIPA buffer (25 mM Tris.HCl pH 7.6, 150 mM NaCl, 1% NP-40, 1% sodium deoxycholate, 0.1% SDS). 20 μg of extract was analyzed by Western blotting on 8% PAGE-SDS using anti-TSC2 antibody (Cell signaling, Tuberin/TSC2 (D93F12) XP® Rabbit mAb #4308).

Flow Cytometry—

Cells were harvested using trypsin, washed with PBS and filtered through 70 μm mesh and analyzed for GFP expression by flow cytometry. Propidium iodide (PI, Sigma) labeling was performed following incubation in serum free media by incubating the cells for 10 minutes with 50 mg/ml PI followed by washing with FACS buffer. Cells were analyzed by either BD FACSort or BD LSRII flow cytometer (Becton Dickinson, Franklin Lakes, N.J.). Data was analyzed using FCS Express V3 analysis software (De Novo, Calif., USA).

Measurement of GFP-Fc Secretion Using the “Typhoon” Fluorescent Scanner—

30×10⁶ HEK293-GFP-Fc cells were harvested using trypsin, washed with PBS and re-suspend in 3 ml of phenol red-free medium in a 50 ml falcon tube. 300 μl were removed and served as control time 0. The Falcon tube was placed in a shaker pre-heated to 37° C. and rotated at 170 rpm. At specific time points following incubation a 300 μl samples were removed. The removed 300 μl samples from each time point were centrifuged at 3000 rpm, 3 minutes at 4° C. and the supernatants were stored protected from light at 4° C. until analysis. For analysis, 100 μl were transferred to black 96 wells plates with UV transparent bottoms (Greiner, #655096). Plates were scanned at excitation 485 nm, emission 528 nm using a “Typhoon FLA7000” fluorscent scanner (GE healthcare) and fluorescence was quantified by imageJ.

Results:

Generation of 293T and CHO Cells Expressing a Reporter for Protein Secretion:

A secretable GFP was constructed by fusing it to the Fc portion of the human IgG1. Upon expression with an MHC class I signal peptide, the GFP-Fc is directed into the endoplasmic reticulum (ER) where it folds and dimerizes via the Fc domain. The resulted protein is then transported from the ER for secretion.

Following cloning of the vector, HEK293 and Chinese Hamster Ovarian (CHO) cells were transfected and subjected to sorting. As can be seen in FIG. 17A, two steps of sorting yielded over 90% CHO cells stably expressing the secretable GFP. Similar results were obtained with HEK293 cells (FIG. 17B).

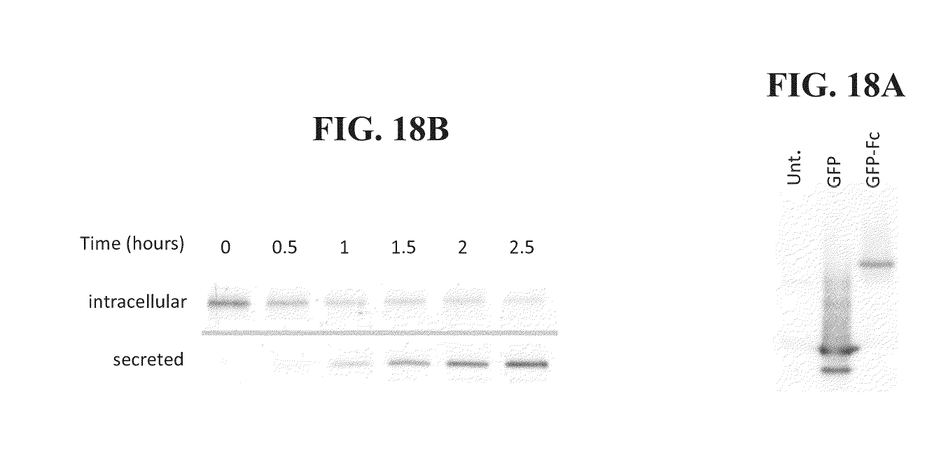

The synthesis and secretion of the GFP-Fc protein was verified by western blot (FIG. 18A) and by pulse chase analysis (FIG. 18B). As clearly demonstrated in FIG. 18B, the yields of GFP-Fc secretion by the transfected HEK293 cells are high. Similar results were obtained in CHO cells (data not shown).

Generation of HEK293-GFP-Fc TSC2 KO and CHO-GFP-Fc TSC2 KO Cells:

In order to generate the knockout of TSC2 in HEK293 and CHO cells a CRISPR vector directed against the TSC2 gene was generated. Following cloning of the vector, HEK293 and CHO cells that stably express the GFP-Fc construct were transfected by two consecutive transfections.

The Effect of TSC2 KO on GFP-Fc Production by HEK293-GFP-Fc Cells:

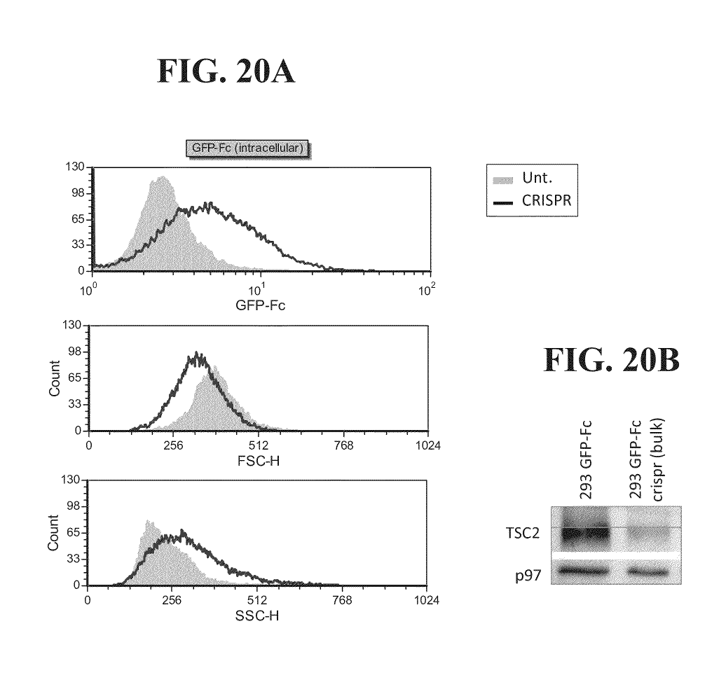

Flow cytometry analysis performed a week following the second transfection of the vector against the TSC2 gene into the HEK293-GFP-Fc cells showed increase in the levels of GFP as well as the cells' side scatter (FIG. 20A). This suggests that the cells synthesize higher levels of the GFP-Fc protein and the content of intracellular granules was elevated.

Western blot analysis showed a significant reduction in the expression of TSC2 in the transfected cells (FIG. 20B).

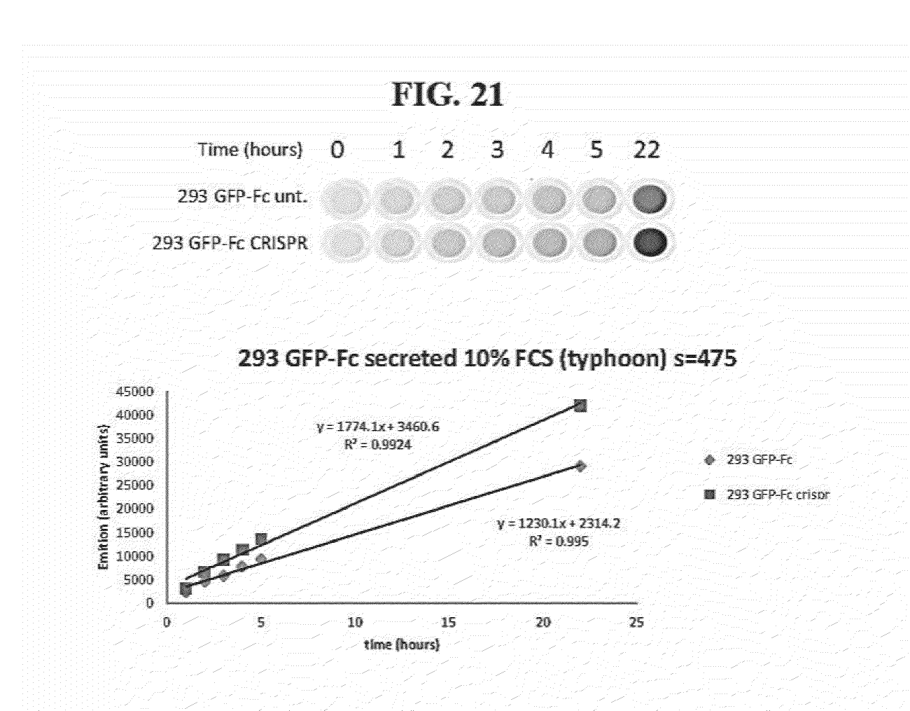

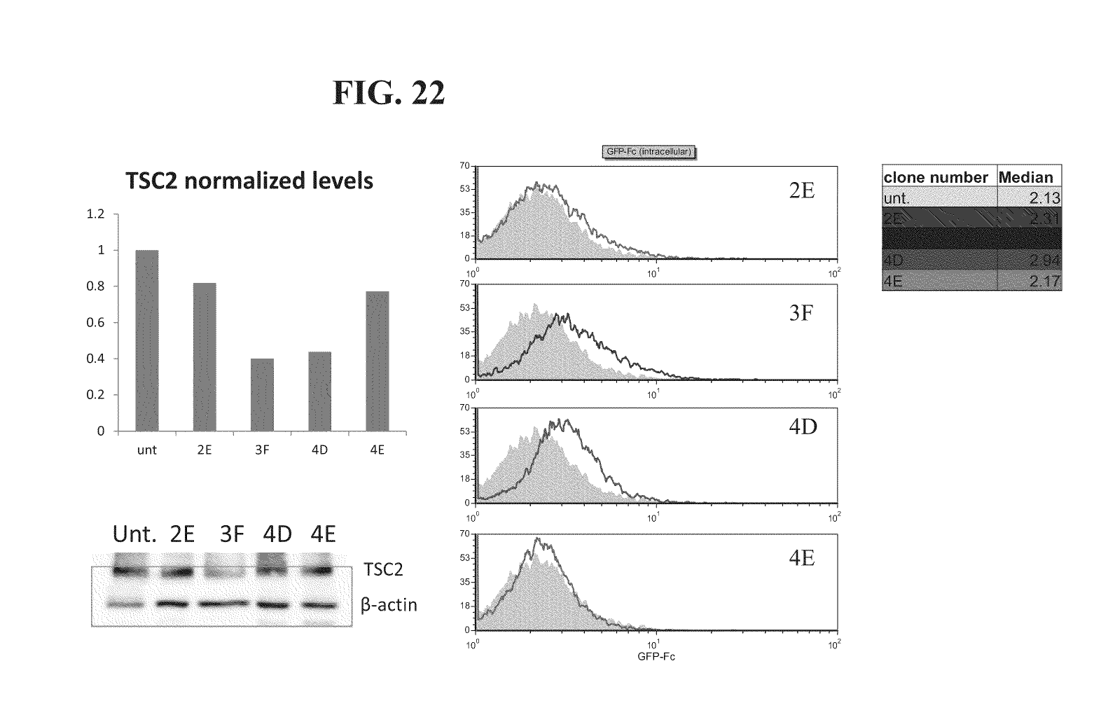

To assess whether the increased content of GFP-Fc also results in an increased secretion, a fluorescent assay using the “Typhoon” fluorescent scanner was developed. As demonstrated in FIG. 21, the rate of GFP accumulation was higher in cells transfected with the vector against the TSC2 gene versus the untransfected controls. Most importantly, as can be seen in FIG. 22, HEK293-GFP-Fc clone 3F displayed the lowest expression of TSC2 and the highest expression of GFP-Fc. This suggests that the decrease in the levels of TSC2 directly correlates with the levels of GFP-Fc synthesis.

The Effect of TSC2 KO on GFP-Fc Production by CHO-GFP-Fc Cells

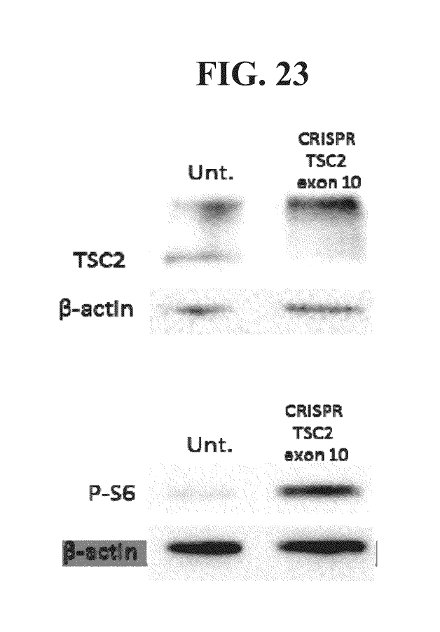

Western blot analysis showed a significant specific reduction in the expression of TSC2 (FIG. 23). This reduction was accompanied by induction of P-S6 levels, indicative for increased mTOR activity (FIG. 23).

TSC2 KO does not Compromise CHO-GFP-Fc Cells Viability

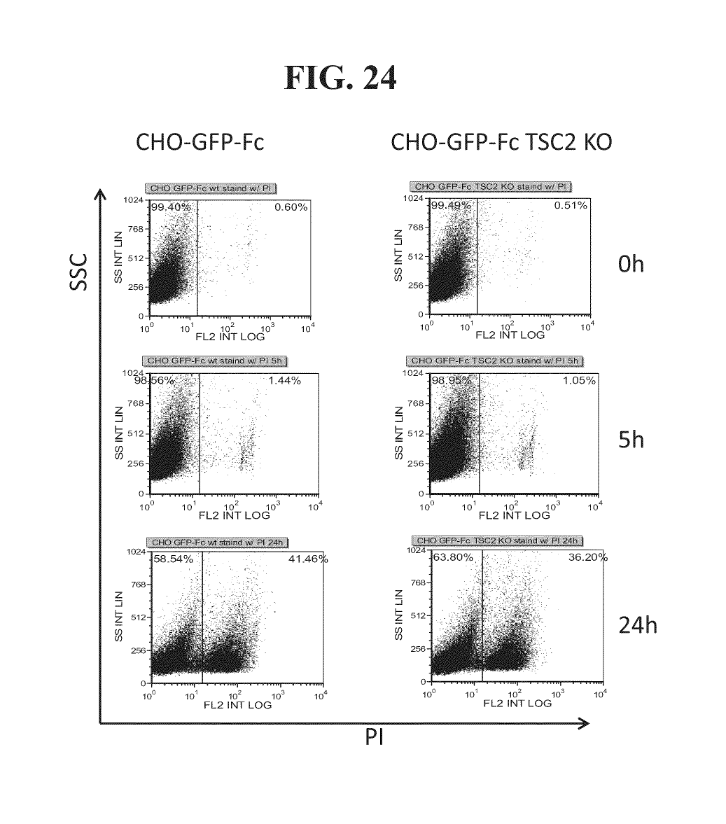

To test whether deletion of TSC2 affect cell viability CHO-GFP-Fc cells and their TSC2 KO derivatives were cultured in serum free media. As can be seen in FIG. 24, following up to 24 hours in culture there was no evident effect on cell viability by deletion of TSC2.

Taken together, this data indicate that deletion of TSC2 increases recombinant GFP-Fc production and secretion by HEK293 and CHO cells without compromising cell viability.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Ron D & Walter P (2007) Signal integration in the endoplasmic reticulum unfolded protein response. Nat Rev Mol Cell Biol 8(7):519-529.
2. Aragon I V, Barrington R A, Jackowski S, Mori K, & Brewer J W (2012) The specialized unfolded protein response of B lymphocytes: ATF6alpha-independent development of antibody-secreting B cells. Mol Immunol 51(3-4):347-355.
3. Gass J N, Jiang H Y, Wek R C, & Brewer J W (2008) The unfolded protein response of B-lymphocytes: PERK-independent development of antibody-secreting cells. Mol Immunol 45(4): 1035-1043.
4. Iwakoshi N N, et al. (2003) Plasma cell differentiation and the unfolded protein response intersect at the transcription factor XBP-1. Nat Immunol 4(4):321-329.
5. Taubenheim N, et al. (2012) High rate of antibody secretion is not integral to plasma cell differentiation as revealed by XBP-1 deficiency. J Immunol 189(7):3328-3338.
6. Tirosh B, Iwakoshi N N, Glimcher L H, & Ploegh H L (2005) XBP-1 specifically promotes IgM synthesis and secretion, but is dispensable for degradation of glycoproteins in primary B cells. J Exp Med 202(4):505-516.
7. Sarbassov D D, et al. (2006) Prolonged rapamycin treatment inhibits mTORC2 assembly and Akt/PKB. Mol Cell 22(2):159-168.
8. Sengupta S, Peterson T R, & Sabatini D M (2010) Regulation of the mTOR complex 1 pathway by nutrients, growth factors, and stress. Mol Cell 40(2):310-322.
9. Kamada Y, et al. (2000) Tor-mediated induction of autophagy via an Apg1 protein kinase complex. J Cell Biol 150(6):1507-1513.
10. Menon S & Manning B D (2008) Common corruption of the mTOR signaling network in human tumors. Oncogene 27 Suppl 2:S43-51.
11. Powell J D & Delgoffe G M (2010) The mammalian target of rapamycin: linking T cell differentiation, function, and metabolism. Immunity 33(3):301-311.
12. Donahue A C & Fruman D A (2007) Distinct signaling mechanisms activate the target of rapamycin in response to different B-cell stimuli. Eur J Immunol 37(10):2923-2936.
13. Ozcan U, et al. (2008) Loss of the tuberous sclerosis complex tumor suppressors triggers the unfolded protein response to regulate insulin signaling and apoptosis. Mol Cell 29(5):541-551.
14. Weichhart T, et al. (2008) The TSC-mTOR signaling pathway regulates the innate inflammatory response. Immunity 29(4):565-577.
15. Goldfinger M, Shmuel M, Benhamron S, & Tirosh B (2011) Protein synthesis in plasma cells is regulated by crosstalk between endoplasmic reticulum stress and mTOR signaling. Eur J Immunol 41(2):491-502.
16. Benhamron S & Tirosh B (2011) Direct activation of mTOR in B lymphocytes confers impairment in B-cell maturation andloss of marginal zone B cells. Eur J Immunol 41(8):2390-2396.
17. Moser K, et al. (2006) Long-lived plasma cells in immunity and immunopathology. Immunol Lett 103(2): 83-85.
18. Tezuka H, et al. (2011) Prominent role for plasmacytoid dendritic cells in mucosal T cell-independent IgA induction. Immunity 34(2):247-257.
19. Mijimolle N, et al. (2005) Protein farnesyltransferase in embryogenesis, adult homeostasis, and tumor development. Cancer Cell 7(4):313-324.
20. Pengo N, et al. (2013) Plasma cells require autophagy for sustainable immunoglobulin production. Nat Immunol 14(3):298-305.
21. Wrammert J, Kallberg E, Agace W W, & Leanderson T (2002) Ly6C expression differentiates plasma cells from other B cell subsets in mice. Eur J Immunol 32(1):97-103.
22. Goldfinger M, et al. (2009) De novo ceramide synthesis is required for N-linked glycosylation in plasma cells. Journal of immunology 182(11):7038-7047.
23. Hu C C, Dougan S K, McGehee A M, Love J C, & Ploegh H L (2009) XBP-1 regulates signal transduction, transcription factors and bone marrow colonization in B cells. Embo J 28(11):1624-1636.
24. Fu S, et al. (2011) Aberrant lipid metabolism disrupts calcium homeostasis causing liver endoplasmic reticulum stress in obesity. Nature 473(7348):528-531.
25. Janes M R, et al. (2010) Effective and selective targeting of leukemia cells using a TORC1/2 kinase inhibitor. Nat Med 16(2):205-213.

1. A method of expressing a recombinant secreted polypeptide of interest, the method comprising:
providing a cell having been contacted with an agent which downregulates an expression of a tuberous sclerosis (TSC) protein or directly inhibits an activity of same; and
contacting the cell with a polynucleotide encoding the secreted recombinant polypeptide of interest.

2. A method of expressing a recombinant secreted polypeptide of interest, the method comprising contacting a cell with:
(i) an agent which downregulates expression of a tuberous sclerosis (TSC) protein or directly inhibits an activity of same;
(ii) a polynucleotide encoding the recombinant secreted polypeptide of interest.

3. A method of increasing production of a recombinant secreted polypeptide of interest, the method comprising contacting a cell which comprises a polynucleotide encoding the recombinant secreted polypeptide of interest with an agent which downregulates an expression of a tuberous sclerosis (TSC) protein or directly inhibits an activity of same, thereby increasing production of the recombinant secreted polypeptide of interest.

4-7. (canceled)

8. An isolated cell having been contacted with an agent which downregulates an expression of a tuberous sclerosis (TSC) protein or directly inhibits an activity of same, wherein the cell further comprises a modified carbohydrate synthesis pathway, glutamine synthetase (GS) and/or dihydrofolate reductase (DHFR) as compared to a control cell of the same species.

9. The method of claim 1, further comprising isolating said recombinant secreted polypeptide.

10. The method of claim 1, further comprising contacting said cell with an agent which downregulates an activity and/or expression of a pro-apoptotic gene.

11. The method of claim 1, further comprising contacting said cell with an agent which upregulates an activity and/or expression of an anti-apoptotic gene.

12-16. (canceled)

17. An isolated cell comprising an exogenous agent which downregulates expression of a tuberous sclerosis (TSC) protein or directly inhibits an activity of same and expressing a recombinant secreted polypeptide of interest.

18. (canceled)

19. The isolated cell of claim 17, further comprising an exogenous agent which upregulates an activity and/or expression of an anti-apoptotic gene.

20. The method of claim 10, wherein said pro-apoptotic gene is selected from the group consisting of BAX, BAK and PUMA.

21. The method of claim 11, wherein said anti-apoptotic gene is selected from the group consisting of Bcl-2, Bcl-xL, Bcl-w, Mcl-1 and XIAP.

22. The method of claim 1, wherein said cell is a mammalian cell.

23. The method of claim 22, wherein said mammalian cell is selected from the group consisting of a Chinese Hamster Ovary (CHO), HEK293, PER.C6, HT1080, NS0, Sp2/0, BHK, Namalwa, COS, HeLa and Vero cell.

24-25. (canceled)

26. The method of claim 1, wherein said secreted polypeptide comprises an antibody or an antibody fragment.

27-29. (canceled)

30. The isolated cell of claim 8 being a cell line.

31. A cell culture comprising the isolated cell of claim 8 and a cell culture medium.

32-34. (canceled)

35. The method of claim 1, wherein said agent is a polynucleotide.

36. The method of claim 1, wherein said agent is a RNA silencing agent.

37. The method of claim 1, wherein said agent is a site specific recombinase.

38. The method of claim 1, wherein said agent is an engineered endonuclease for genome editing.

39. The method of claim 35, wherein said polynucleotide is selected from the group consisting of an antisense, siRNA, miRNA, zinc finger nuclease, CRISPR/Cas and TALEN.

40. The method of claim 1, wherein said agent comprises a polynucleotide sequence selected from the group consisting of SEQ ID NOs. 20-28.

41. The method of claim 1, wherein said agent interferes with the formation of a TSC1/TSC2 complex.

42. The method of claim 1, wherein said agent binds to and/or cleaves said TSC.

43. The method of claim 1, wherein said agent is selected from the group consisting of an aptamer, a small molecule, an inhibitory peptide, antibody and antibody fragment.

44. The method of claim 1, wherein said agent increases phosphorylation of S6.

45. The method of claim 1-44, wherein said tuberous sclerosis is TSC1 or TSC2.

46. (canceled)


SEQUENCE LISTING (42 sequences)

SEQ ID NO: 1 - protein, 1161 aa, Cricetulus griseus
MAQLANIGELLSMLDSPTLGVRDDVTTIFKESLNSERGPMLVNTLVDYYLETNSQPVLHILTTLQEPHDKHLLDKINEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPSEASYEDGYSVSHQLSACFPHRSADVTASPYVDTQNSYGGTTSTPSSTSRLMLFSPAGQLPQSLSSPSTRLLPEPLQATHWSPSMVCGMTTPPTSPGNVPPELSHPYSKAFSTTGGKGTPLGTPATSPPPALPCPPDDCVHGPSSQATATPPRKEERADSSRPYLQRQQYLLNDRGLEDPPASKGSVTLGNLPDFLGDLASEEDSIEKDKEEAAISKELSEITTAEADPVVPRGGFDSPFYRDSLSSSQRKTHSAASGTQGSSVNPEPLHSSLDKHGPDTPKQAFTPIDPPSGSADASPAGDRDRQTSLETSILTPSPCKIPPQRGVSFGSGQPPPYDHLFEVALPKTACHFVSKKTEELLKKVKGTPDEDCVPSTSPMEVLDRLIEQGADAHSKELSKLSLPSKSVDWTHFGGSPPSDEIRTLRDQLLLLHNQLLYERFKRQQHALRNRRLLRKVIRAAALEEHNAAMKDQLKLQEKDIQMWKLSLEKEQDRYSQLQDQHDTMVTQLHSQIRQLQHDREEFYNQSQELQTKLEDCRNMIAELRVELKKANNKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLRKLEEDKAETAEAAEERLDCCSDGCSDSLVGHNEEASGHNGEARTTRPSGPRASCGGRSTGGSSSSSSELSTPEKPPSQRFNSRWETTVGEPSGSIPTTVGSLPSSKSFLGMKARELFRNKSESQCDEDCVTGSSLSETLKTELGRDSGMENKTPFNLDASHPSSPNSDSVGQLHIMDYNETHQEHS

SEQ ID NO: 2 - protein, 1156 aa, Cricetulus griseus
MAQLANIGELLSMLDSPTLGVRDDVTTIFKESLNSERGPMLVNTLVDYYLETNSQPVLHILTTLQEPHDKHLLDKINEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPSEASYEDGYSVSHQLSACFPHRSADVTASPYVDTQNSYGGTTSTPSSTSRLMLFSPAGQLPQSLSSPSTRLLPEPLQATHWSPSMVCGMTTPPTSPGNVPPELSHPYSKAFSTTGGKGTPLGTPATSPPPALPCPPDDCVHGPSSQATATPPRKEERADSSRPYLQRQQYLLNDRGLEDPPASKGSVTLGNLPDFLGDLASEEDSIEKDKEEAAISKELSEITTAEADPVVPRGGFDSPFYRDSLSSSQRKTHSAASGTQGSSVNPEPLHSSLDKHGPDTPKQAFTPIDPPSGSADASPAGDRDRQTSLETSILTPSPCKIPPQRGVSFGSGQPPPYDHLFEVALPKTACHFVSKKTEELLKKVKGTPDEDCVPSTSPMEVLDRLIEQGADAHSKELSKLSLPSKSVDWTHFGDEIRTLRDQLLLLHNQLLYERFKRQQHALRNRRLLRKVIRAAALEEHNAAMKDQLKLQEKDIQMWKLSLEKEQDRYSQLQDQHDTMVTQLHSQIRQLQHDREEFYNQSQELQTKLEDCRNMIAELRVELKKANNKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLRKLEEDKAETAEAAEERLDCCSDGCSDSLVGHNEEASGHNGEARTTRPSGPRASCGGRSTGGSSSSSSELSTPEKPPSQRFNSRWETTVGEPSGSIPTTVGSLPSSKSFLGMKARELFRNKSESQCDEDCVTGSSLSETLKTELGRDSGMENKTPFNLDASHPSSPNSDSVGQLHIMDYNETHQEHS

SEQ ID NO: 3 - protein, 1169 aa, Cricetulus griseus
  misc_feature (703)..(706): Xaa can be any naturally occurring amino acid
MAQLANIGELLSMLDSPTLGVRDDVTTIFKESLNSERGPMLVNTLVDYYLETNSQPVLHILTTLQEPHDKHLLDKINEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPSEASYEDGYSVSHQLSACFPHRSADVTASPYVDTQNSYGGTTSTPSSTSRLMLFSPAGQLPQSLSSPSTRLLPEPLQATHWSPSMVCGMTTPPTSPGNVPPELSHPYSKAFSTTGGKGTPLGTPATSPPPALPCPPDDCVHGPSSQATATPPRKEERADSSRPYLQRQQYLLNDRGLEDPPASKGSVTLGNLPDFLGDLASEEDSIEKDKEEAAISKELSEITTAEADPVVPRGGFDSPFYRDSLSSSQRKTHSAASGTQGSSVNPEPLHSSLDKHGPDTPKQAFTPIDPPSGSADASPAGDRDRQTSLETSILTPSPCKIPPQRGVSFGSGQPPPYDHLFEVALPKTACHFVSKKTEELLKKVKGTPDEDCVPSTSPMEVLDRLIEQGADAHSKELSKLSLPSKSVDWTHFGGSPPSDEIRTLRDQLLLLHNQLLXXXXLLKWIKRFKRQQHALRNRRLLRKVIRAAALEEHNAAMKDQLKLQEKDIQMWKLSLEKEQDRYSQLQDQHDTMVTQLHSQIRQLQHDREEFYNQSQELQTKLEDCRNMIAELRVELKKANNKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLRKLEEDKAETAEAAEERLDCCSDGCSDSLVGHNEEASGHNGEARTTRPSGPRASCGGRSTGGSSSSSSELSTPEKPPSQRFNSRWETTVGEPSGSIPTTVGSLPSSKSFLGMKARELFRNKSESQCDEDCVTGSSLSETLKTELGRDSGMENKTPFNLDASHPSSPNSDSVGQLHIMDYNETHQEHS

SEQ ID NO: 4 - protein, 1160 aa, Mus musculus
MAQLANIGELLSMLDSSTLGVRDDVTAIFKESLNSERGPMLVNTLVDYYLETNSQPVLHILTTLQEPHDKHLLDKINEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPTEASYEDGYSVSHQLSACFPYRSADVTTSPYVDTQNSYGGSTSTPSSSSRLMLFSPPGQLPQSLSSPSTRLLPEPLQASLWSPSAVCGMTTPPTSPGNVPADLSHPYSKAFGTTGGKGTPSGTPATSPPPAPPCPQDDCVHGSAAQASATAPRKEERADSSRPYLHRQSNDRGLEDPPGSKGSVTLRNLPDFLGDLASEEDSIEKDKEEAAISKELSEITTAEADPVVPRGGFDSPFYRDSLSGSQRKTHSAASGTQGSSVNPEPLHSSLDKHGPDTPKQAFTPIDPPSGSADVSPAGDRDRQTSLETSILTPSPCKIPPQRGVSFGSGQLPPYDHLFEVALPKTACHFVSKKTEELLKKVKGNPEEDCVPSTSPMEVLDRLIEQGAGAHSKELSRLSLPSKSVDWTHFGGSPPSDELRTLRDQLLLLHNQLLYERFKRQQHALRNRRLLRKVIRAAALEEHNAAMKDQLKLQEKDIQMWKVSLQKEQARYSQLQEQRDTMVTQLHSQIRQLQHDREEFYNQSQELQTKLEDCRNMIAELRVELKKANNKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLRKLEEDRAEAAEAAEERLDCCSDGCTDSLVGHNEEASGHNGETRTSRPGGTRASCGGRVTGGSSSSSSELSTPEKPPSQRFSSRWEPALGEPSSSIPTTVGSLPSSKSFLGMKARELFRNKSESQCDEDSVTMSSSSLSETLKTELGKDSGTENKTSLSLDAPHPSSPNSDNVGQLHIMDYNETHPEHS

SEQ ID NO: 5 - protein, 1164 aa, Homo sapiens
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 6 - protein, 1163 aa, Homo sapiens
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 7 - protein, 1113 aa, Homo sapiens
MAQQANVGELLAMLDSPMLGVRDDVTAVFKENLNSDRGPMLVNTLVDYYLETSSQPALHILTTLQEPHDKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 8 - protein, 1785 aa, Mus musculus
MAKPTSKDSGLKEKFKILLGLGTSRPNPRCAEGKQTEFIITSEILRELSGECGLNNRIRMIGQICDVAKTKKLEEHAVEALWKAVSDLLQPERPPEARHAVLTLLKAIVQGQGDRLGVLRALFFKVIKDYPSNEDLHERLEVFKALTDNGRHITYLEEELAEFVLQWMDVGLSSEFLLVLVNLVKFNSCYLDEYIASMVHMICLLCIRTVSSVDIEVSLQVLDAVVCYNCLPAESLPLFIITLCRTINVKELCEPCWKLMRNLLGTHLGHSAIYNMCRIMEDRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIHPAKDGWIQNLQLLMERFFRNECRSAVRIKVLDVLSFVLLINRQFYEEELINSVVISQLSHIPEDKDHQVRKLATQLLVDLAEGCHTHHFNSLLDIIEKVMARSLSPPPELEERDLAMHSASLEDVKTAVLGLLVILQTKLYTLPASHATRVYESLISHIQLHYKHGYSLPIASSIRLQAFDFLLLLRADSLHRLGLPNKDGVVRFSPYCLCDCMELDRASEKKASGPLSPPTGPPSPVPMGPAVRLGYLPYSLLFRVLLQCLKQESDWKVLKLVLSRLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLLSFDDTPEKDSFRARSTSLNERPKSLRIARAPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSDSSSDPSTHVRQTKEAPAKLESQAGQQVSRGARDRVRSMSGGHGLRVGVLDTSAPYSPGGSASLGPQTAVAAKPEKPPAGAQLPTAEKTNLAAYVPLLTQGWAEILVRRPTGNTSWLMSLENPLSPFSSDINNMPLQELSNALMAAERFKEHRDTALYKSLSVPAAGTAKPPTLPRSNTDSAMVLEEGSPGETQVPVEPPELEDFEAALGTDRHCQRPDTYSRSSSASSQEEKSHLEELAAGGIPIERAISSEGARPAVDLSFQPSQPLSKSSSSPELQTLQDILGDLGDKIDIGRLSPEAKVRSQSGILDGEAATWSATGEESRITVPPEGPLPSSSPRSPSGLRPRGYTISDSAPSRRGKRVERDNFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRQRIREEVHYSNPSLPLMHPPAHTKAPAQAPEATPTYETGQRKRLISSVDDFTEFV

SEQ ID NO: 9 - protein, 1742 aa, Mus musculus
MAKPTSKDSGLKEKFKILLGLGTSRPNPRCAEGKQTEFIITSEILRELSGECGLNNRIRMIGQICDVAKTKKLEEHAVEALWKAVSDLLQPERPPEARHAVLTLLKAIVQGQGDRLGVLRALFFKVIKDYPSNEDLHERLEVFKALTDNGRHITYLEEELAEFVLQWMDVGLSSEFLLVLVNLVKFNSCYLDEYIASMVHMICLLCIRTVSSVDIEVSLQVLDAVVCYNCLPAESLPLFIITLCRTINVKELCEPCWKLMRNLLGTHLGHSAIYNMCRIMEDRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIHPAKDGWIQNLQLLMERFFRNECRSAVRIKVLDVLSFVLLINRQFYEEELINSVVISQLSHIPEDKDHQVRKLATQLLVDLAEGCHTHHFNSLLDIIEKVMARSLSPPPELEERDLAMHSASLEDVKTAVLGLLVILQTKLYTLPASHATRVYESLISHIQLHYKHGYSLPIASSIRLQAFDFLLLLRADSLHRLGLPNKDGVVRFSPYCLCDCMELDRASEKKASGPLSPPTGPPSPVPMGPAVRLGYLPYSLLFRVLLQCLKQESDWKVLKLVLSRLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLLSFDDTPEKDSFRARSTSLNERPKSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSDSSSDPSTHVRQTKEAPAKLESQAGQQVSRGARDRVRSMSGGHGLRVGVLDTSAPYSPGGSASLGPQTAVAAKPEKPPAGAQLPTAEKTNLAAYVPLLTQGWAEILVRRPTGNTSWLMSLENPLSPFSSDINNMPLQELSNALMAAERFKEHRDTALYKSLSVPAAGTAKPPTLPRSNTDSAMVLEEGSPGETQVPVEPPELEDFEAALGTDRHCQRPDTYSRSSSASSQEEKSHLEELAAGGIPIERAISSEGARPAVDLSFQPSQPLSKSSSSPELQTLQDILGDLGDKIDIGRLSPEAKVRSQSGILDGEAATWSATGEESRITVPPEGPLPSSSPRSPSGLRPRGYTISDSAPSRRGKRVERDNFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRQRIREEVHYSNPSLPLMHPPAHTKAPAQAPEATPTYETGQRKRLISSVDDFTEFV

SEQ ID NO: 10 - protein, 1808 aa, Mus musculus
MAKPTSKDSGLKEKFKILLGLGTSRPNPRCAEGKQTEFIITSEILRELSGECGLNNRIRMIGQICDVAKTKKLEEHAVEALWKAVSDLLQPERPPEARHAVLTLLKAIVQGQGDRLGVLRALFFKVIKDYPSNEDLHERLEVFKALTDNGRHITYLEEELAEFVLQWMDVGLSSEFLLVLVNLVKFNSCYLDEYIASMVHMICLLCIRTVSSVDIEVSLQVLDAVVCYNCLPAESLPLFIITLCRTINVKELCEPCWKLMRNLLGTHLGHSAIYNMCRIMEDRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIHPAKDGWIQNLQLLMERFFRNECRSAVRIKVLDVLSFVLLINRQFYEEELINSVVISQLSHIPEDKDHQVRKLATQLLVDLAEGCHTHHFNSLLDIIEKVMARSLSPPPELEERDLAMHSASLEDVKTAVLGLLVILQTKLYTLPASHATRVYESLISHIQLHYKHGYSLPIASSIRLQAFDFLLLLRADSLHRLGLPNKDGVVRFSPYCLCDCMELDRASEKKASGPLSPPTGPPSPVPMGPAVRLGYLPYSLLFRVLLQCLKQESDWKVLKLVLSRLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLLSFDDTPEKDSFRARSTSLNERPKSLRIARAPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSDSSSDPSTHVRQTKEAPAKLESQAGQQVSRGARDRVRSMSGGHGLRVGVLDTSAPYSPGGSASLGPQTAVAAKPEKPPAGAQLPTAEKTNLAAYVPLLTQGWAEILVRRPTGNTSWLMSLENPLSPFSSDINNMPLQELSNALMAAERFKEHRDTALYKSLSVPAAGTAKPPTLPRSNTVASFSSLYQPSCQGQLHRSVSWADSAMVLEEGSPGETQVPVEPPELEDFEAALGTDRHCQRPDTYSRSSSASSQEEKSHLEELAAGGIPIERAISSEGARPAVDLSFQPSQPLSKSSSSPELQTLQDILGDLGDKIDIGRLSPEAKVRSQSGILDGEAATWSATGEESRITVPPEGPLPSSSPRSPSGLRPRGYTISDSAPSRRGKRVERDNFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRQRIREEVHYSNPSLPLMHPPAHTKAPAQAPEATPTYETGQRKRLISSVDDFTEFV

SEQ ID NO: 11 - protein, 1741 aa, Mus musculus
MAKPTSKDSGLKEKFKILLGLGTSRPNPRCAEGKQTEFIITSEILRELSGECGLNNRIRMIGQICDVAKTKKLEEHAVEALWKAVSDLLQPERPPEARHAVLTLLKAIVQGQGDRLGVLRALFFKVIKDYPSNEDLHERLEVFKALTDNGRHITYLEEELAEFVLQWMDVGLSSEFLLVLVNLVKFNSCYLDEYIASMVHMICLLCIRTVSSVDIEVSLQVLDAVVCYNCLPAESLPLFIITLCRTINVKELCEPCWKLMRNLLGTHLGHSAIYNMCRIMEDRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIHPAKDGWIQNLQLLMERFFRNECRSAVRIKVLDVLSFVLLINRQFYEEELINSVVISQLSHIPEDKDHQVRKLATQLLVDLAEGCHTHHFNSLLDIIEKVMARSLSPPPELEERDLAMHSASLEDVKTAVLGLLVILQTKLYTLPASHATRVYESLISHIQLHYKHGYSLPIASSIRLQAFDFLLLLRADSLHRLGLPNKDGVVRFSPYCLCDCMELDRASEKKASGPLSPPTGPPSPVPMGPAVRLGYLPYSLLFRVLLQCLKQESDWKVLKLVLSRLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLLSFDDTPEKDSFRARSTSLNERPKRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSDSSSDPSTHVRQTKEAPAKLESQAGQQVSRGARDRVRSMSGGHGLRVGVLDTSAPYSPGGSASLGPQTAVAAKPEKPPAGAQLPTAEKTNLAAYVPLLTQGWAEILVRRPTGNTSWLMSLENPLSPFSSDINNMPLQELSNALMAAERFKEHRDTALYKSLSVPAAGTAKPPTLPRSNTDSAMVLEEGSPGETQVPVEPPELEDFEAALGTDRHCQRPDTYSRSSSASSQEEKSHLEELAAGGIPIERAISSEGARPAVDLSFQPSQPLSKSSSSPELQTLQDILGDLGDKIDIGRLSPEAKVRSQSGILDGEAATWSATGEESRITVPPEGPLPSSSPRSPSGLRPRGYTISDSAPSRRGKRVERDNFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRQRIREEVHYSNPSLPLMHPPAHTKAPAQAPEATPTYETGQRKRLISSVDDFTEFV

SEQ ID NO: 12 - protein, 1814 aa, Mus musculus
MAKPTSKDSGLKEKFKILLGLGTSRPNPRCAEGKQTEFIITSEILRELSGECGLNNRIRMIGQICDVAKTKKLEEHAVEALWKAVSDLLQPERPPEARHAVLTLLKAIVQGQGDRLGVLRALFFKVIKDYPSNEDLHERLEVFKALTDNGRHITYLEEELAEFVLQWMDVGLSSEFLLVLVNLVKFNSCYLDEYIASMVHMICLLCIRTVSSVDIEVSLQVLDAVVCYNCLPAESLPLFIITLCRTINVKELCEPCWKLMRNLLGTHLGHSAIYNMCRIMEDRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIHPAKDGWIQNLQLLMERFFRNECRSAVRIKVLDVLSFVLLINRQFYEEELINSVVISQLSHIPEDKDHQVRKLATQLLVDLAEGCHTHHFNSLLDIIEKVMARSLSPPPELEERDLAMHSASLEDVKTAVLGLLVILQTKLYTLPASHATRVYESLISHIQLHYKHGYSLPIASSIRLQAFDFLLLLRADSLHRLGLPNKDGVVRFSPYCLCDCMELDRASEKKASGPLSPPTGPPSPVPMGPAVRLGYLPYSLLFRVLLQCLKQESDWKVLKLVLSRLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLLSFDDTPEKDSFRARSTSLNERPKSLRIARAPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSDSSSDPSTHVRQTKEAPAKLESQAGQQVSRGARDRVRSMSGGHGLRVGVLDTSAPYSPGGSASLGPQTAVAAKPEKPPAGAQLPTAEKTNLAAYVPLLTQGWAEILVRRPTGNTSWLMSLENPLSPFSSDINNMPLQELSNALMAAERFKEHRDTALYKSLSVPAAGTAKPPTLPRSNTVASFSSLYQPSCQGQLHRSVSWADSAMVLEEGSPGETQVPVEPPELEDFEAALGTDRHCQRPDTYSRSSSASSQEEKSHLEELAAGGIPIERAISSEGARPAVDLSFQPSQPLSKSSSSPELQTLQDILGDLGDKIDIGRLSPEAKVRSQSGILDGEAATWSATGEESRITVPPEGPLPSSSPRSPSGLRPRGYTISDSAPSRRGKRVERDNFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDGPACKCEWWRQPGEIVVWALPVVMELTVTILLCHLQMASQVHHSRSNPTDIYPSKWIARLRHIKRLRQRIREEVHYSNPSLPLMHPPAHTKAPAQAPEATPTYETGQRKRLISSVDDFTEFV

SEQ ID NO: 13 - protein, 1786 aa, Cricetulus griseus
MAKPASKDSGLKEKFKILLGLGTSRPNPRCAEGKQTEFIITAEILRELSGECGLSNRIRMIGQICDVAKTKKFEEHAVEALWKAVSDLLQPERPPEARHAVLALLKAIVQGQGDRLGVLRALFFKVIKDYPSNEDLHERLEVFKALTDNGKHITYLEEELAEFVLQWMDVGLSSEFLLVLVNLVKFNSCYLDEYIASMVHMICLLCIQTVSSVDIEVSLKVLDAVVCYNCLPAESLPLFIITLCRTINVKELCEPCWKLMRNLLGTHLGHSAIYNMCRIMESRSYMEDAPLLRGAVFFVGMALWGAHRLYSLKNSPTSVLPSFYQAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIYPAKDGWIQNLQLLMDRFFRNECRSAVRIKVLDVLSFVLLINRQFYEEELINSVVISQLSHIPEDKDHQVRKLATQLLVDLAEGCHTHHFNSLLDIIEKVMARSLSPPLELEERDVAVYSASLEDVKTAVLGLLVILQTKLYTLPASHATRVYETLISHIQLHYKHGYSLPIASSIRLQAFDFLLLLRADSLHRLGLPNKDGVVRFSPYCLCDCMELERASEKKASGPLSPPTGPPSPVPTGPAVRLGYLPYSLLFRVLLQCLKQETDWKVLKLVLSKLPESLRYKVLIFTSPCNVDQLSSALCSMLSDPKTLERLRGTPEGFSRTDLHLAVVPVLTALISYHNYLDKTKQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLMSFDDTPEKDSFRARSTSLNERPKSLRIARVPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQSGPESSSDSGVRVRQTKEAPAKLESQAGQQVSRGARDRVRSMSGGHGLRVGALDTTAPHTSGGPASLGPQTAPATKPEKASAGTQLPKAETTNLAAYVPLLTQGWAEILVRRPTGNTSWLMSLENPLSPFSSDINNMPLQELSNALMAAERFKEHRDTALYKSLSVPAAGTAKPPPLPRSNTDSAVVHEEGSPGEAYVPVEPPELEDFESSLGTDRHCQRPDTYSRSSSASSQEEKSHLEELAAGGIPIERAISSEGTRPAVDLSFQPSQTLSKSSSSPELQTLQDILGDLGDKADLGRLSPESKVRSQSGILDGEAATWSAPGEEGRVTVPPEGPLPSSSPRSPNGLRPRGYTISDSAPSRRGKRVERDTFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRHRIREEVHYPNPSLPLMHPPAHTKAPAQAPAESTPTYETGQRKRLISSVDDFTEFV

SEQ ID NO: 14 - protein, 1743 aa, Cricetulus griseus
MAKPASKDSGLKEKFKILLGLGTSRPNPRCAEGKQTEFIITAEILRELSGECGLSNRIRMIGQICDVAKTKKFEEHAVEALWKAVSDLLQPERPPEARHAVLALLKAIVQGQGDRLGVLRALFFKVIKDYPSNEDLHERLEVFKALTDNGKHITYLEEELAEFVLQWMDVGLSSEFLLVLVNLVKFNSCYLDEYIASMVHMICLLCIQTVSSVDIEVSLKVLDAVVCYNCLPAESLPLFIITLCRTINVKELCEPCWKLMRNLLGTHLGHSAIYNMCRIMESRSYMEDAPLLRGAVFFVGMALWGAHRLYSLKNSPTSVLPSFYQAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIYPAKDGWIQNLQLLMDRFFRNECRSAVRIKVLDVLSFVLLINRQFYEEELINSVVISQLSHIPEDKDHQVRKLATQLLVDLAEGCHTHHFNSLLDIIEKVMARSLSPPLELEERDVAVYSASLEDVKTAVLGLLVILQTKLYTLPASHATRVYETLISHIQLHYKHGYSLPIASSIRLQAFDFLLLLRADSLHRLGLPNKDGVVRFSPYCLCDCMELERASEKKASGPLSPPTGPPSPVPTGPAVRLGYLPYSLLFRVLLQCLKQETDWKVLKLVLSKLPESLRYKVLIFTSPCNVDQLSSALCSMLSDPKTLERLRGTPEGFSRTDLHLAVVPVLTALISYHNYLDKTKQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLMSFDDTPEKDSFRARSTSLNERPKSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQSGPESSSDSGVRVRQTKEAPAKLESQAGQQVSRGARDRVRSMSGGHGLRVGALDTTAPHTSGGPASLGPQTAPATKPEKASAGTQLPKAETTNLAAYVPLLTQGWAEILVRRPTGNTSWLMSLENPLSPFSSDINNMPLQELSNALMAAERFKEHRDTALYKSLSVPAAGTAKPPPLPRSNTDSAVVHEEGSPGEAYVPVEPPELEDFESSLGTDRHCQRPDTYSRSSSASSQEEKSHLEELAAGGIPIERAISSEGTRPAVDLSFQPSQTLSKSSSSPELQTLQDILGDLGDKADLGRLSPESKVRSQSGILDGEAATWSAPGEEGRVTVPPEGPLPSSSPRSPNGLRPRGYTISDSAPSRRGKRVERDTFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRHRIREEVHYPNPSLPLMHPPAHTKAPAQAPAESTPTYETGQRKRLISSVDDFTEFV

SEQ ID NO: 15 - protein, 1809 aa, Cricetulus griseus
MAKPASKDSGLKEKFKILLGLGTSRPNPRCAEGKQTEFIITAEILRELSGECGLSNRIRMIGQICDVAKTKKFEEHAVEALWKAVSDLLQPERPPEARHAVLALLKAIVQGQGDRLGVLRALFFKVIKDYPSNEDLHERLEVFKALTDNGKHITYLEEELAEFVLQWMDVGLSSEFLLVLVNLVKFNSCYLDEYIASMVHMICLLCIQTVSSVDIEVSLKVLDAVVCYNCLPAESLPLFIITLCRTINVKELCEPCWKLMRNLLGTHLGHSAIYNMCRIMESRSYMEDAPLLRGAVFFVGMALWGAHRLYSLKNSPTSVLPSFYQAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIYPAKDGWIQNLQLLMDRFFRNECRSAVRIKVLDVLSFVLLINRQFYEEELINSVVISQLSHIPEDKDHQVRKLATQLLVDLAEGCHTHHFNSLLDIIEKVMARSLSPPLELEERDVAVYSASLEDVKTAVLGLLVILQTKLYTLPASHATRVYETLISHIQLHYKHGYSLPIASSIRLQAFDFLLLLRADSLHRLGLPNKDGVVRFSPYCLCDCMELERASEKKASGPLSPPTGPPSPVPTGPAVRLGYLPYSLLFRVLLQCLKQETDWKVLKLVLSKLPESLRYKVLIFTSPCNVDQLSSALCSMLSDPKTLERLRGTPEGFSRTDLHLAVVPVLTALISYHNYLDKTKQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLMSFDDTPEKDSFRARSTSLNERPKSLRIARVPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQSGPESSSDSGVRVRQTKEAPAKLESQAGQQVSRGARDRVRSMSGGHGLRVGALDTTAPHTSGGPASLGPQTAPATKPEKASAGTQLPKAETTNLAAYVPLLTQGWAEILVRRPTGNTSWLMSLENPLSPFSSDINNMPLQELSNALMAAERFKEHRDTALYKSLSVPAAGTAKPPPLPRSNTVASFSSLYQPSCQGQLHRSVSWADSAVVHEEGSPGEAYVPVEPPELEDFESSLGTDRHCQRPDTYSRSSSASSQEEKSHLEELAAGGIPIERAISSEGTRPAVDLSFQPSQTLSKSSSSPELQTLQDILGDLGDKADLGRLSPESKVRSQSGILDGEAATWSAPGEEGRVTVPPEGPLPSSSPRSPNGLRPRGYTISDSAPSRRGKRVERDTFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHSRSNPTDIYPSKWIARLRHIKRLRHRIREEVHYPNPSLPLMHPPAHTKAPAQAPAESTPTYETGQRKRLISSVDDFTEFV

SEQ ID NO: 16 - protein, 1807 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 17 - protein, 1740 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTDSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 18 - protein, 1784 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTDSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 19 - DNA, 24 bp, Artificial Sequence
  Single strand DNA oligonucleotide
aacaccaagatacctgcttgggtc

SEQ ID NO: 20 - DNA, 23 bp, Artificial Sequence
  Single strand DNA oligonucleotide
gtgcaataccggttgagaattgg

SEQ ID NO: 21 - DNA, 23 bp, Artificial Sequence
  Single strand DNA oligonucleotide
gcagatggacaccgacgttgtgg

SEQ ID NO: 22 - DNA, 20 bp, Artificial Sequence
  Single strand DNA oligonucleotide
atgacaagcacctcttggac

SEQ ID NO: 23 - DNA, 20 bp, Artificial Sequence
  Single strand DNA oligonucleotide
ctaccaatgattccacagtc

SEQ ID NO: 24 - DNA, 23 bp, Artificial Sequence
  Single strand DNA oligonucleotide
gtctttagggtgaccgtttgggg

SEQ ID NO: 25 - DNA, 20 bp, Artificial Sequence
  Single strand DNA oligonucleotide
tcttcgtagggatggcactc

SEQ ID NO: 26 - DNA, 23 bp, Artificial Sequence
  Single strand DNA oligonucleotide
gagagccatggaactcgttctgg

SEQ ID NO: 27 - DNA, 20 bp, Artificial Sequence
  Single strand DNA oligonucleotide
cgaagaccttcacgaaaggc

SEQ ID NO: 28 - DNA, 20 bp, Artificial Sequence
  Single strand DNA oligonucleotide
aacaatcgcatccggatgat

SEQ ID NO: 29 - DNA, 21 bp, Artificial Sequence
  Single strand DNA oligonucleotide
gcagtgctacgagtgctatgg

SEQ ID NO: 30 - DNA, 23 bp, Artificial Sequence
  Single strand DNA oligonucleotide
actgacgggtctttagtttcctt

SEQ ID NO: 31 - DNA, 22 bp, Artificial Sequence
  Single strand DNA oligonucleotide
cagccgtatatcttcccagact

SEQ ID NO: 32 - DNA, 23 bp, Artificial Sequence
  Single strand DNA oligonucleotide
ctcagagggatgccagtaatcta

SEQ ID NO: 33 - DNA, 24 bp, Artificial Sequence
  Single strand DNA oligonucleotide
ctttgtggtcgtagggtaggaacc

SEQ ID NO: 34 - DNA, 24 bp, Artificial Sequence
  Single strand DNA oligonucleotide
acttgcaccaacacttgccatttc

SEQ ID NO: 35 - DNA, 24 bp, Artificial Sequence
  Single strand DNA oligonucleotide
caaggtggttcactgcctgtaatg

SEQ ID NO: 36 - DNA, 20 bp, Artificial Sequence
  Single strand DNA oligonucleotide
gactatcatatgcttaccgt

SEQ ID NO: 37 - DNA, 20 bp, Artificial Sequence
  Single strand DNA oligonucleotide
accacctacacacccaccca

SEQ ID NO: 38 - DNA, 20 bp, Artificial Sequence
  Single strand DNA oligonucleotide
gcctcctcccacctcttagt

SEQ ID NO: 39 - DNA, 20 bp, Artificial Sequence
  Single strand DNA oligonucleotide
catcccactctctgccctct

SEQ ID NO: 40 - DNA, 20 bp, Artificial Sequence
  Single strand DNA oligonucleotide
atccctctccaccctcttgc

SEQ ID NO: 41 - DNA, 21 bp, Artificial Sequence
  Single strand DNA oligonucleotide
gtccctctctactctcttgcc

SEQ ID NO: 42 - DNA, 21 bp, Artificial Sequence
  Single strand DNA oligonucleotide
tccctttctaccctctttccc